SEQUENCE LISTING (64 sequences)

SEQ ID NO: 1 - protein, 22 aa, Homo sapiens
MLLLVTSLLLCELPHPAFLLIP

SEQ ID NO: 2 - protein, 108 aa, synthetic construct
  REGION 1..108: note = Synthetic Polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITG

SEQ ID NO: 3 - protein, 17 aa, synthetic construct
  REGION 1..17: note = Synthetic Polypeptide
STSGSGKPGSGEGSTKG

SEQ ID NO: 4 - protein, 120 aa, synthetic construct
  REGION 1..120: note = Synthetic Polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5 - protein, 45 aa, Homo sapiens
TTTPAPRPPTPAPTIASQPLSLRPEACRPAAGGAVHTRGLDFACD

SEQ ID NO: 6 - protein, 26 aa, Homo sapiens
IYIWAPLAGTCGVLLLSLVITLYCWV

SEQ ID NO: 7 - protein, 42 aa, Homo sapiens
KRGRKKLLYIFKQPFMRPVQTTQEEDGCSCRFPEEEEGGCEL

SEQ ID NO: 8 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 21 aa, synthetic construct
  REGION 1..21: note = Synthetic Polypeptide
GSGEGRGSLLTCGDVEENPGP

SEQ ID NO: 10 - protein, 21 aa, Homo sapiens
MALPVTALLLPLALLLHAARP

SEQ ID NO: 11 - protein, 111 aa, synthetic construct
  REGION 1..111: note = Synthetic Polypeptide
DIVLTQSPASLAVSLGQRATISCKASQSVDYDGDSYMNWYQQKPGQPPKLLIYVASNLESGIPARFSGSGSGTDFTLNIHPVEEEDAATYYCQQSNEDPFTFGSGTKLEIN

SEQ ID NO: 12 - protein, 15 aa, synthetic construct
  REGION 1..15: note = Synthetic Polypeptide
GGGGSGGGGSGGGGS

SEQ ID NO: 13 - protein, 119 aa, synthetic construct
  REGION 1..119: note = Synthetic Polypeptide
EVQLQESGAELVKPGASVKMSCKASGYTFTSYWMHWVKQRPGQGLEWIGAIDPSDSYTGYNQKFKGKATLTVDTSSSTAYMHLSSLTSEDSAVYFCTRSYYGNSWFAYWGQGTLVTVSA

SEQ ID NO: 14 - protein, 37 aa, Homo sapiens
RRDQRLPPDAHKPPGGGSFRTPIQEEQADAHSTLAKI

SEQ ID NO: 15 - protein, 999 aa, synthetic construct
  REGION 1..999: note = Synthetic Polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPDIVLTQSPASLAVSLGQRATISCKASQSVDYDGDSYMNWYQQKPGQPPKLLIYVASNLESGIPARFSGSGSGTDFTLNIHPVEEEDAATYYCQQSNEDPFTFGSGTKLEINGGGGSGGGGSGGGGSEVQLQESGAELVKPGASVKMSCKASGYTFTSYWMHWVKQRPGQGLEWIGAIDPSDSYTGYNQKFKGKATLTVDTSSSTAYMHLSSLTSEDSAVYFCTRSYYGNSWFAYWGQGTLVTVSATSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 66 bp, Homo sapiens
  source 1..66: mol_type = genomic DNA
atgctgctgctcgtgacctccctgctgctgtgcgagctgccacaccctgccttcctgctgatccct

SEQ ID NO: 17 - DNA, 324 bp, synthetic construct
  misc_feature 1..324: note = Synthetic Polynucleotide
  source 1..324: mol_type = other DNA
gacatccagatgacccagaccacaagctccctgtccgcctctctgggcgacagagtgacaatctcttgtagggccagccaggatatctccaagtatctgaactggtaccagcagaagccagatggcaccgtgaagctgctgatctatcacacatctaggctgcacagcggagtgccatcccggtttagcggatccggatctggaaccgactactctctgacaatcagcaacctggagcaggaggatatcgccacctatttctgccagcagggcaataccctgccttacacatttggcggcggcacaaagctggagatcaccggc

SEQ ID NO: 18 - DNA, 51 bp, synthetic construct
  misc_feature 1..51: note = Synthetic Polynucleotide
  source 1..51: mol_type = other DNA
agcacatccggatctggcaagccaggatccggagagggatctaccaaggga

SEQ ID NO: 19 - DNA, 360 bp, synthetic construct
  misc_feature 1..360: note = Synthetic Polynucleotide
  source 1..360: mol_type = other DNA
gaggtgaagctgcaggagagcggaccaggactggtggcacccagccagtccctgtctgtgacctgtacagtgtccggcgtgtctctgccagactacggcgtgagctggatcaggcagccacctaggaagggactggagtggctgggcgtgatctggggctccgagaccacatactataatagcgccctgaagtccagactgaccatcatcaaggataacagcaagtcccaggtgttcctgaagatgaattccctgcagaccgacgatacagccatctactattgcgccaagcactactattacggcggctcctatgccatggactactggggccagggcacctctgtgacagtgtctagc

SEQ ID NO: 20 - DNA, 135 bp, Homo sapiens
  source 1..135: mol_type = genomic DNA
accacgacaccggctccaagaccacccactcccgcgcccacaatcgcttcccaaccattgagccttaggcctgaggcgtgccgaccggcagcgggtggagctgtacacactagggggctcgactttgcgtgtgac

SEQ ID NO: 21 - DNA, 78 bp, Homo sapiens
  source 1..78: mol_type = genomic DNA
atttatatctgggcgccattggccggtacttgtggtgtattgctcctctcccttgttattacattgtattgctgggtg

SEQ ID NO: 22 - DNA, 126 bp, Homo sapiens
  source 1..126: mol_type = genomic DNA
aaacgaggtagaaaaaaacttctttatatattcaaacaaccatttatgagaccagtacaaactactcaagaggaagatggatgtagttgtcgatttccagaagaagaagaaggaggatgtgaactg

SEQ ID NO: 23 - DNA, 336 bp, Homo sapiens
  source 1..336: mol_type = genomic DNA
agggtaaaatttagtagatccgccgacgccccggcttaccagcagggtcagaatcaactctataacgagctgaacctcgggcgcagagaagagtacgacgtcttggataagcggagagggcgagaccctgaaatggggggaaaaccgcgacggaagaatcctcaagagggactctacaacgagttgcagaaggacaaaatggcggaagcgtacagtgagataggaatgaaaggagaacgcagacgcggtaagggccatgacgggctctaccagggcctgtcaacagctacaaaagatacatacgacgcccttcatatgcaggcgctgccaccacgg

SEQ ID NO: 24 - DNA, 63 bp, synthetic construct
  misc_feature 1..63: note = Synthetic Polynucleotide
  source 1..63: mol_type = other DNA
ggttctggagagggaaggggctctctgctgacatgcggcgatgtggaggagaatcctggccca

SEQ ID NO: 25 - DNA, 63 bp, Homo sapiens
  source 1..63: mol_type = genomic DNA
atggccctgcctgtgacagccctgctgctgcctctggctctgctgctgcatgccgctagaccc

SEQ ID NO: 26 - DNA, 333 bp, synthetic construct
  misc_feature 1..333: note = Synthetic Polynucleotide
  source 1..333: mol_type = other DNA
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatttatgttgcatccaatctagaatctggaatcccagccaggtttagtggcagtgggtctgggacagacttcaccctcaacatccatcctgtggaggaggaggatgctgcaacctattactgtcagcaaagtaatgaggacccattcacgttcggctcggggacaaagttggaaataaac

SEQ ID NO: 27 - DNA, 45 bp, synthetic construct
  misc_feature 1..45: note = Synthetic Polynucleotide
  source 1..45: mol_type = other DNA
ggaggaggtggtagtggtggaggaggaagtggaggaggaggaagt

SEQ ID NO: 28 - DNA, 357 bp, synthetic construct
  misc_feature 1..357: note = Synthetic Polynucleotide
  source 1..357: mol_type = other DNA
gaggtgcagctgcaggagtctggggctgagctggtgaagcctggggcttcagtgaagatgtcctgcaaggcttctggctacaccttcaccagctactggatgcactgggtgaagcagaggcctggacaaggccttgagtggatcggagcaattgatccttcagatagttatactggctacaatcaaaagttcaagggcaaggccacattgactgtagacacatcctccagcacagcctacatgcacctcagcagcctgacatctgaggactctgcggtctatttctgtacaagaagctactatggtaactcctggtttgcttactggggccaagggactctggtcactgtctctgca

SEQ ID NO: 29 - DNA, 111 bp, Homo sapiens
  source 1..111: mol_type = genomic DNA
cggagggaccagaggctgccccccgatgcccacaagccccctgggggaggcagtttccggacccccatccaagaggagcaggccgacgcccactccaccctggccaagatc

SEQ ID NO: 30 - DNA, 3000 bp, synthetic construct
  misc_feature 1..3000: note = Synthetic Polynucleotide
  source 1..3000: mol_type = other DNA
atgctgctgctcgtgacctccctgctgctgtgcgagctgccacaccctgccttcctgctgatccctgacatccagatgacccagaccacaagctccctgtccgcctctctgggcgacagagtgacaatctcttgtagggccagccaggatatctccaagtatctgaactggtaccagcagaagccagatggcaccgtgaagctgctgatctatcacacatctaggctgcacagcggagtgccatcccggtttagcggatccggatctggaaccgactactctctgacaatcagcaacctggagcaggaggatatcgccacctatttctgccagcagggcaataccctgccttacacatttggcggcggcacaaagctggagatcaccggcagcacatccggatctggcaagccaggatccggagagggatctaccaagggagaggtgaagctgcaggagagcggaccaggactggtggcacccagccagtccctgtctgtgacctgtacagtgtccggcgtgtctctgccagactacggcgtgagctggatcaggcagccacctaggaagggactggagtggctgggcgtgatctggggctccgagaccacatactataatagcgccctgaagtccagactgaccatcatcaaggataacagcaagtcccaggtgttcctgaagatgaattccctgcagaccgacgatacagccatctactattgcgccaagcactactattacggcggctcctatgccatggactactggggccagggcacctctgtgacagtgtctagcaccacgacaccggctccaagaccacccactcccgcgcccacaatcgcttcccaaccattgagccttaggcctgaggcgtgccgaccggcagcgggtggagctgtacacactagggggctcgactttgcgtgtgacatttatatctgggcgccattggccggtacttgtggtgtattgctcctctcccttgttattacattgtattgctgggtgaaacgaggtagaaaaaaacttctttatatattcaaacaaccatttatgagaccagtacaaactactcaagaggaagatggatgtagttgtcgatttccagaagaagaagaaggaggatgtgaactgagggtaaaatttagtagatccgccgacgccccggcttaccagcagggtcagaatcaactctataacgagctgaacctcgggcgcagagaagagtacgacgtcttggataagcggagagggcgagaccctgaaatggggggaaaaccgcgacggaagaatcctcaagagggactctacaacgagttgcagaaggacaaaatggcggaagcgtacagtgagataggaatgaaaggagaacgcagacgcggtaagggccatgacgggctctaccagggcctgtcaacagctacaaaagatacatacgacgcccttcatatgcaggcgctgccaccacggggttctggagagggaaggggctctctgctgacatgcggcgatgtggaggagaatcctggcccaatggccctgcctgtgacagccctgctgctgcctctggctctgctgctgcatgccgctagacccgacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatttatgttgcatccaatctagaatctggaatcccagccaggtttagtggcagtgggtctgggacagacttcaccctcaacatccatcctgtggaggaggaggatgctgcaacctattactgtcagcaaagtaatgaggacccattcacgttcggctcggggacaaagttggaaataaacggaggaggtggtagtggtggaggaggaagtggaggaggaggaagtgaggtgcagctgcaggagtctggggctgagctggtgaagcctggggcttcagtgaagatgtcctgcaaggcttctggctacaccttcaccagctactggatgcactgggtgaagcagaggcctggacaaggccttgagtggatcggagcaattgatccttcagatagttatactggctacaatcaaaagttcaagggcaaggccacattgactgtagacacatcctccagcacagcctacatgcacctcagcagcctgacatctgaggactctgcggtctatttctgtacaagaagctactatggtaactcctggtttgcttactggggccaagggactctggtcactgtctctgcaactagtacaactactccagcaccacgaccaccaacacctgctccaactatcgcatctcaaccactttctctacgtccagaagcatgccgaccagctgcaggaggtgcagttcatacgagaggtctagatttcgcatgtgatatctacatctgggcaccattggctgggacttgtggtgtccttctcctatcactggttatcaccctttactgccggagggaccagaggctgccccccgatgcccacaagccccctgggggaggcagtttccggacccccatccaagaggagcaggccgacgcccactccaccctggccaagatcagggtgaagttttctcgcagcgccgatgccccagcctatcagcagggccagaaccagctgtacaacgagctgaatctgggcaggcgcgaggagtacgacgtgctggataagaggagaggaagggatccagagatgggaggcaagcctaggcgcaagaacccacaggagggcctgtataatgagctgcagaaggacaagatggccgaggcctacagcgagatcggcatgaagggagagaggagaaggggcaagggacacgatggcctgtatcagggcctgtccacagccaccaaggacacctacgatgcactgcacatgcaggcactgccacctagataa

SEQ ID NO: 31 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 32 - DNA, 2118 bp, Homo sapiens
  source 1..2118: mol_type = genomic DNA
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 33 - protein, 706 aa, synthetic construct
  REGION 1..706: note = Synthetic Polypeptide
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGLEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 34 - DNA, 2118 bp, synthetic construct
  misc_feature 1..2118: note = Synthetic Polynucleotide
  source 1..2118: mol_type = other DNA
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcttgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 35 - DNA, 357 bp, synthetic construct
  misc_feature 1..357: note = Synthetic Polynucleotide
  source 1..357: mol_type = other DNA
gaggtgcagctgcaggagtctggggctgagctggtgaagcctggggcttcagtgaagatgtcctgcaaggcttctggctacaccttcaccagctactggatgcactgggtgaagcagaggcctggaccaggccttgagtggatcggagcaattgatccttcagatagttatactggctacaatcaaaagttcaagggcaaggccacattgactgtagacacatcctccagcacagcctacatgcacctcagcagcctgacatctgaggactctgcggtctatttctgtacaagaagctactatggtaactcctggtttgcttactggggccaagggactctggtcactgtctctgca

SEQ ID NO: 36 - protein, 119 aa, synthetic construct
  REGION 1..119: note = Synthetic Polypeptide
EVQLQESGAELVKPGASVKMSCKASGYTFTSYWMHWVKQRPGPGLEWIGAIDPSDSYTGYNQKFKGKATLTVDTSSSTAYMHLSSLTSEDSAVYFCTRSYYGNSWFAYWGQGTLVTVSA

SEQ ID NO: 37 - protein, 8 aa, synthetic construct
  REGION 1..8: note = Synthetic Polypeptide
GYTFTSYW

SEQ ID NO: 38 - protein, 8 aa, synthetic construct
  REGION 1..8: note = Synthetic Polypeptide
IDPSDSYT

SEQ ID NO: 39 - protein, 13 aa, synthetic construct
  REGION 1..13: note = Synthetic Polypeptide
NSWFAYWGQGTLV

SEQ ID NO: 40 - DNA, 333 bp, synthetic construct
  misc_feature 1..333: note = Synthetic Polynucleotide
  source 1..333: mol_type = other DNA
acattgtgctgacccaatctccagcttctttggctgtgtctctagggcagagggccaccatctcctgcaaggccagccaaagtgttgattatgatggtgatagttatataaactggtaccaacagaaaccaggacagccacccaaactcctcatctatgctgcatccaatctagaatctggaatcccagccaggtttagtgccagtgggtctgggacagacttcaccctcaacatccatcctgtggaggaggaggatgttgcagcctattactgtcagcaaagtaatgaggacccattcacgttcggctcggggacaaggttggaaataaaac

SEQ ID NO: 41 - protein, 110 aa, synthetic construct
  REGION 1..110: note = Synthetic Polypeptide
IVLTQSPASLAVSLGQRATISCKASQSVDYDGDSYINWYQQKPGQPPKLLIYAASNLESGIPARFSASGSGTDFTLNIHPVEEEDVAAYYCQQSNEDPFTFGSGTRLEIK

SEQ ID NO: 42 - protein, 10 aa, synthetic construct
  REGION 1..10: note = Synthetic Polypeptide
QSVDYDGDSY

SEQ ID NO: 43 - skipped

SEQ ID NO: 44 - protein, 9 aa, synthetic construct
  REGION 1..9: note = Synthetic Polypeptide
QQSNEDPFT

SEQ ID NO: 45 - DNA, 357 bp, synthetic construct
  misc_feature 1..357: note = Synthetic Polynucleotide
  source 1..357: mol_type = other DNA
gaggtgcagctgcaggagtctggggctgagctggtgaagcctggggcttcagtgaagatgtcctgcaaggcttctggctacaccttcaccagctactggatgcactgggtgaagcagaggcctggacaaggccttgagtggatcggagcaattgatccttcagatagttatactggctacaatcaaaagttcaagggcaaggccacattgactgtagacacatcctccagcacagcctacatgcacctcagcagcctgacatctgaggactctgcggtctatttctgtacaagaagctactatggtaactcctggtttgattactggggccaagggactctggtcactgtctctgca

SEQ ID NO: 46 - protein, 119 aa, synthetic construct
  REGION 1..119: note = Synthetic Polypeptide
EVQLQESGAELVKPGASVKMSCKASGYTFTSYWMHWVKQRPGQGLEWIGAIDPSDSYTGYNQKFKGKATLTVDTSSSTAYMHLSSLTSEDSAVYFCTRSYYGNSWFDYWGQGTLVTVSA

SEQ ID NO: 47 - protein, 8 aa, synthetic construct
  REGION 1..8: note = Synthetic Polypeptide
GYTFTSYW

SEQ ID NO: 48 - protein, 8 aa, synthetic construct
  REGION 1..8: note = Synthetic Polypeptide
IDPSDSYT

SEQ ID NO: 49 - protein, 13 aa, synthetic construct
  REGION 1..13: note = Synthetic Polypeptide
NSWFDYWGQGTLV

SEQ ID NO: 50 - DNA, 333 bp, synthetic construct
  misc_feature 1..333: note = Synthetic Polynucleotide
  source 1..333: mol_type = other DNA
gacattgtgctgacccaatctccagcttctttggctgtgtctctagggcagagggccaccatctcctgcaaggccagccaaagtgttgattatgaaggtgatagttatatgaactggtaccaacagaaaccaggacagccacccaaactcctcatctatgctgcatccaatctagaatctggaatcccagccaggtttagtggcagtgggtctgggacagacttcaccctcaacatccatcctgtggaggaggaggatgctgcaacctatcactgtcagcaaagtaatgaggacccgttcacgttcggaggggggaccaagttggaaataaaa

SEQ ID NO: 51 - protein, 111 aa, synthetic construct
  REGION 1..111: note = Synthetic Polypeptide
DIVLTQSPASLAVSLGQRATISCKASQSVDYEGDSYMNWYQQKPGQPPKLLIYAASNLESGIPARFSGSGSGTDFTLNIHPVEEEDAATYHCQQSNEDPFTFGGGTKLEIK

SEQ ID NO: 52 - protein, 10 aa, synthetic construct
  REGION 1..10: note = Synthetic Polypeptide
QSVDYEGDSY

SEQ ID NO: 53 - skipped

SEQ ID NO: 54 - protein, 9 aa, synthetic construct
  REGION 1..9: note = Synthetic Polypeptide
QQSNEDPFT

SEQ ID NO: 55 - DNA, 357 bp, synthetic construct; identical to SEQ ID NO: 28
  misc_feature 1..357: note = Synthetic Polynucleotide
  source 1..357: mol_type = other DNA

SEQ ID NO: 56 - protein, 119 aa, synthetic construct; identical to SEQ ID NO: 13
  REGION 1..119: note = Synthetic Polypeptide

SEQ ID NO: 57 - protein, 8 aa, synthetic construct
  REGION 1..8: note = Synthetic Polypeptide
GYTFTSYW

SEQ ID NO: 58 - protein, 7 aa, synthetic construct
  REGION 1..7: note = Synthetic Polypeptide
DPSDSYT

SEQ ID NO: 59 - protein, 12 aa, synthetic construct
  REGION 1..12: note = Synthetic Polypeptide
SWFAYWGQGTLV

SEQ ID NO: 60 - DNA, 333 bp, synthetic construct; identical to SEQ ID NO: 26
  misc_feature 1..333: note = Synthetic Polynucleotide
  source 1..333: mol_type = other DNA

SEQ ID NO: 61 - protein, 111 aa, synthetic construct; identical to SEQ ID NO: 11
  REGION 1..111: note = Synthetic Polypeptide

SEQ ID NO: 62 - protein, 10 aa, synthetic construct
  REGION 1..10: note = Synthetic Polypeptide
QSVDYDGDSY

SEQ ID NO: 63 - skipped

SEQ ID NO: 64 - protein, 9 aa, synthetic construct
  REGION 1..9: note = Synthetic Polypeptide
QQSNEDPFT

(19) United States
(12) Patent Application Publication
(10) Pub. No.: US 2025/0161447 A1
(43) Pub. Date: May 22, 2025

(54) ANTI-CD19 AND ANTI-CD79B CHIMERIC ANTIGEN RECEPTORS AND METHODS OF USE THEREOF

(71) Applicant: BOARD OF REGENTS, THE UNIVERSITY OF TEXAS SYSTEM, Austin, TX (US)

(72) Inventors: Sattva S. NEELAPU, Houston, TX (US); Xiaoyun CHENG, Houston, TX (US); Jingjing CAO, Houston, TX (US); Fuliang CHU, Houston, TX (US)

(73) Assignee: BOARD OF REGENTS, THE UNIVERSITY OF TEXAS SYSTEM, Austin, TX (US)

(21) Appl. No.: 18/840,450

(22) PCT Filed: Mar. 3, 2023
(86) PCT No.: PCT/US2023/063721

§ 371 Date: Aug. 21, 2024

Related U.S. Application Data
(60) Provisional application No. 63/316,311, filed on Mar. 3, 2022.

(51) Int. Cl. A61K 40/31; A61K 40/42; A61P 35/00; C07K 14/705; C07K 14/725; C07K 16/28; C12N 5/078

(52) U.S. Cl. CPC A61K 40/31; A61K 40/4211; A61K 40/4224; A61P 35/00; C07K 14/70503; C07K 14/7051; C07K 14/70517; C07K 14/70596; C07K 16/2803; C12N 5/0634; A61K 2239/13; A61K 2239/21; A61K 2239/22; A61K 2239/28; A61K 2239/48; C07K 2317/56; C07K 2317/622; C12N 2510/00

(57) ABSTRACT

Aspects of the present disclosure describe dual chimeric antigen receptor (CAR) immune cells, as well as methods for making such cells and for use in treatment of cancer. Disclosed are immune cells comprising both anti-CD19 and anti-CD79b CARs and methods for use in treatment of B-cell malignancies. Also disclosed are polynucleotides encoding an anti-CD19 CAR and an anti-CD79 CAR, as well as cells comprising such polynucleotides and pharmaceutical compositions comprising such cells.

This application claims priority to U.S. Provisional Patent Application Ser. No. 63/316,311, filed Mar. 3, 2022, which is incorporated by reference herein in its entirety.

### SEQUENCE LISTING

The application contains a Sequence Listing which has been filed electronically in compliance with ST.26 format and is hereby incorporated by reference in its entirety. Said Sequence Listing, created on Mar. 3, 2023 is named MDAC.P1328WO.xml and is 87,011 bytes in size.

### BACKGROUND

### I. Technical Field

This disclosure relates generally at least to the fields of cancer biology, immunology, and medicine.

### II. Background

Recent studies demonstrated that chimeric antigen receptor (CAR)-modified T cell therapy targeting CD19 induced high response rates in the majority of patients with refractory B-cell malignancies including follicular lymphoma (FL), mantle cell lymphoma (MCL), diffuse large B-cell lymphoma (DLBCL), and chronic lymphocytic leukemia (CLL).¹ A subset of these patients experience long-term remissions suggesting adoptive T-cell therapy could be curative in patients refractory to existing therapies. However, >50% of patients relapse or progress after CD19 CAR T-cell therapy, and a major cause of failure appears to be due to the loss of CD19 expression on the tumor cells.^{2,3} Thus, there is a need for development of CAR immune cell (e.g., CAR T-cell) therapies that minimize immune escape and improve efficacy over existing CAR T-cell therapies.

### SUMMARY

Described herein, in some aspects, are dual CAR immune cells targeting at least CD19 and CD79b, and methods of use thereof, including for the treatment of cancer. Also disclosed are polynucleotides encoding anti-CD19 and anti-CD79b CARs, along with methods for generating engineered immune cells comprising such polynucleotides. In some aspects, a polynucleotide of the disclosure is, or is a portion of, a bicistronic vector expressing both an anti-CD19 CAR and an anti-CD79b CAR, in some cases separated by one or more elements that allow the anti-CD19 CAR and anti-CD79b CAR to be expressed as separate molecules. In specific embodiments, the element is a self-cleaving peptide such as a 2A element. Further aspects of the disclosure are described herein.

Embodiments of the present disclosure include nucleic acids, polynucleotides, polypeptides, proteins, peptides, constructs, vectors, cells, therapeutic cells, immune cells, engineered cells, methods for generating engineered cells, methods for detecting engineered cells, methods for isolating engineered cells, methods for depleting engineered cells, and methods for purifying engineered cells. Nucleic acids of the disclosure may encode one or more polypeptides of the disclosure, including one or more chimeric polypeptides. In some embodiments, a nucleic acid molecule of the disclosure encodes a chimeric polypeptide. In some embodiments, a nucleic acid molecule of the disclosure encodes two or more chimeric polypeptides. A chimeric polypeptide of the disclosure can include at least 1, 2, 3, or more of the following regions or domains: a signal peptide, an extracellular domain, a hinge region, a transmembrane domain, and an intracellular region. An engineered cell of the disclosure can comprise 1, 2, 3, 4, or more polynucleotides and/or polypeptides of the disclosure. Methods of the present disclosure can include at least 1, 2, 3, 4, or more of the following steps: introducing a polynucleotide into a cell, introducing a vector into a cell, introducing a polypeptide into a cell, expressing a polypeptide in a cell, expanding a population of cells, contacting a cell with an antigen-binding protein, contacting a cell with an antibody drug conjugate, and detecting a cell with an imaging agent.

Disclosed herein, in some aspects, is a polynucleotide comprising (a) a first sequence encoding an anti-CD19 chimeric antigen receptor (CAR), the anti-CD19 CAR comprising: (i) a GM-CSF Receptor alpha signal peptide; (ii) a CD19-binding domain; (iii) a CD8α hinge region; (iv) a CD8α transmembrane domain; (v) a 4-1BB signaling domain; and (vi) a CD3 zeta signaling domain; and (b) a second sequence encoding an anti-CD79b CAR, the anti-CD79b CAR comprising: (i) a CD8α signal peptide; (ii) a CD79b-binding domain; (iii) a CD8α hinge region; (iv) a CD8α transmembrane domain; (v) a OX40 signaling domain; and (vi) a CD3 zeta signaling domain. In some aspects, the first sequence is 5′ relative to the second sequence. In some aspects, the polynucleotide further comprises a third sequence encoding a T2A peptide. In some aspects, the third sequence is 3′ relative to the first sequence and 5′ relative to the second sequence. In some aspects, the T2A peptide comprises SEQ ID NO:9.

In some aspects, the GM-CSF Receptor signal peptide comprises SEQ ID NO:1. In some aspects, the CD19-binding domain is an scFv of an anti-CD19 antibody. In some aspects, the CD19-binding domain comprises a VL having SEQ ID NO:2 and a VH having SEQ ID NO: 4. In some aspects, the CD19-binding domain further comprises a linker linking the VL and the VH. In some aspects, the linker comprises SEQ ID NO:3. In some aspects, the CD8α hinge region of (a) comprises SEQ ID NO:5. In some aspects, the CD8α transmembrane domain of (a) comprises SEQ ID NO:6. In some aspects, the 4-1BB signaling domain comprises SEQ ID NO:7. In some aspects, the CD3 zeta signaling domain of (a) comprises SEQ ID NO:8.

In some aspects, the CD8α signal peptide comprises SEQ ID NO:10. In some aspects, the CD79b-binding domain is an scFv of an anti-CD79 antibody. In some aspects, the CD79b-binding domain comprises a VL having SEQ ID NO:11 and a VH having SEQ ID NO: 13. In some aspects, the CD79b-binding domain further comprises a linker linking the VL and the VH. In some aspects, the CD79b-binding domain comprises a linker having SEQ ID NO: 12. In some aspects, the CD8α hinge region of (b) comprises SEQ ID NO:5. In some aspects, the CD8α transmembrane domain of (b) comprises SEQ ID NO:6. In some aspects, the OX40 signaling domain comprises SEQ ID NO: 14. In some aspects, the CD3 zeta signaling domain of (b) comprises SEQ ID NO:8.

In some aspects, the polynucleotide comprises a sequence encoding a polypeptide having SEQ ID NO:15. In some aspects, the polynucleotide comprises a sequence having at least 90%, 91%, 92%, 93%, 94%, 95%, 96%, 97%, 98%, 99%, or 99.9% identity with SEQ ID NO: 30, or any range or value derivable therein. In some aspects, the polynucleotide further comprises a promoter. In some aspects, the promoter is an EF1α promoter.

Further disclosed is a vector comprising a polynucleotide disclosed herein. Also provided is a polypeptide encoded by a polynucleotide or vector disclosed herein. Further described is a method of generating an engineered cell comprising introducing into the cell a polynucleotide, vector, or polypeptide disclosed herein.

Also disclosed, in some aspects, is an engineered cell comprising a polynucleotide, vector, or polypeptide disclosed herein. In some aspects, the engineered cell is a T cell. In some aspects, the engineered cell is a natural killer (NK) cell. Also contemplated herein is a population of engineered cells comprising an engineered cell disclosed herein. In some aspects, at least 35% of the engineered cells in the population express both the anti-CD19 CAR and the anti-CD79b CAR. In some aspects, the population of engineered cells comprises a subset that express either the anti-CD19 CAR or the anti-CD79b CAR, wherein at least 80% of the subset express both the anti-CD19 CAR and the anti-CD79b CAR. Further contemplated is a pharmaceutical composition comprising a population of engineered cells disclosed herein.

Also described, in some aspects, is a method for treating a subject for cancer, the method comprising administering to the subject a therapeutically effective amount of a pharmaceutical composition disclosed herein (e.g., a pharmaceutical composition comprising a population of engineered cells such as, for example, dual CAR immune cells). In some aspects, the subject has a B-cell malignancy. In some aspects, the subject has diffuse large B-cell lymphoma, high-grade B-cell lymphoma, follicular lymphoma, marginal zone lymphoma, mantle cell lymphoma, Burkitt lymphoma, lymphoplasmacytic lymphoma, hairy cell leukemia, or chronic lymphocytic leukemia. In some aspects, the subject has CD19⁺CD79b⁺ cancer.

Also disclosed, in some aspects, is a polynucleotide encoding a polypeptide having SEQ ID NO:15. In some aspects, the polynucleotide comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 99.9% identity with SEQ ID NO:30, or any range or value derivable therein. In some aspects, the polynucleotide comprises SEQ ID NO:30.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B show an example dual CD19−CD79b CAR construct and expression analysis. FIG. 1A shows a schematic of a dual CD19−CD79b CAR construct composed of anti-CD19 CAR and anti-CD79b CAR cloned into a third-generation lentiviral vector under the control of EF1α promoter. The T2A self-cleaving peptide links the two CARs and facilitates their simultaneous expression. FIG. 1B shows expression of CD19 and CD79b CARs on T cells transduced with the dual CAR construct shown in FIG. 1A, which was assessed by staining with FITC-conjugated CD19 protein and PE-conjugated CD79b protein that bind to CD19 and CD79b scFvs, respectively.

FIGS. 2A-2G show results demonstrating that dual CD19−CD79b CAR T cells degranuate in CD19 or CD79b dependent manner. FIG. 2A shows that dual CD19−CD79b CD4+ CAR T cells degranulate in response to CD19+CD79b+ tumor cells. Untransduced T, CD19 CAR T, and dual CD19−CD79b CAR T were co-cultured with B-cell lymphoma cell lines (CD19+CD79b+: Daudi, SUDHL6, PDX203, and PDX300) for 6 hours at 1:2 effector:target (E:T) ratio. Flow cytometry plots show high CD107a expression in CD4+subset of CD19 CAR and dual CD19−CD79b CAR T cells. FIG. 2B shows that dual CD19−CD79b CD8+ CAR T cells degranulate in response to CD19+CD79b+ tumor cells. Experiment was set-up as sssdescribed for FIG. 2A, and CD107a expression in CD8+ subset is shown. FIG. 2C shows that dual CD19−CD79b CAR T cells degranulate in CD19 dependent manner. Untransduced T, CD19 CAR T, and dual CD19−CD79b CAR T cells were co-cultured with B-cell leukemia cell line, NALM6, that is CD19+CD79b−for 6 hours at 1:1 effector:target (E:T) ratio. CD107a expression in both CD4+and CD8+T cells is shown. FIG. 2D shows that dual CD19−CD79b CAR T cells degranulate in CD79b dependent manner. Untransduced T, CD19 CAR T, and dual CD19−CD79b CAR T cells were co-cultured with SUDHL6−CD19KO cell line for 6 hours at 1:1 effector:target (E:T) ratio. CD107a expression in both CD4+and CD8+T cells is shown. FIG. 2E shows that dual CD19−CD79b CAR T cells are not responsive to CD19−CD79b− tumor cells. Untransduced T, CD19 CAR T, and dual CD19−CD79b CAR T cells were co-cultured with CD19−CD79b− K562 tumor cells for 6 hours at 1:1 effector:target (E:T) ratio and CD107a expression was assessed by flow cytometry. FIG. 2F shows that cryopreserved and thawed dual CD19−CD79b CAR T cells degranulate in CD79b dependent manner. Thawed untransduced T cells, CD19 CAR T cells, CD79b CAR T cells, and CD19−CD79b dual CAR T cells were co-cultured with CD19KO B-cell lymphoma tumor cells (Daudi-CD19KO and19KO) for 6 hours at 2:1 effector:target (E:T) ratio and CD107a expression was assessed by flow cytometry. FIG. 2G shows that cryopreserved and thawed dual CD19−CD79b CAR T cells degranulate in response to CD19+CD79b+ Daudi tumor cells. Thawed untransduced T cells, CD19 CAR T cells, CD79b CAR T cells, and CD19−CD79b dual CAR T cells were co-cultured with Daudi tumor cells for 6 hours at 1:1 effector:target (E:T) ratio and CD107a expression was assessed by flow cytometry.

FIGS. 3A-3I show results demonstrating that dual CAR T cells mediate specific lysis of tumor cells in CD19 or CD79b dependent manner. FIG. 3A shows that dual CAR T cells induced lysis of CD19+CD79b+tumors. Untransduced T, CD19 CAR T, and dual CD19−CD79b CAR T cells were co-cultured with CD19+CD79b+B-cell lymphoma cell lines (Daudi, SUDHL6, PDX203, and PDX300) for 96 hours at 1:1 effector:target (E:T) ratio. Percentages of live tumor cells on day 0 and day 4 are shown for each co-culture condition. FIG. 3B shows that dual CAR T cells induced lysis of CD19+CD79b− tumor cells. Untransduced T and dual CD19−CD79b CAR T cells were co-cultured with CD19+CD79b− B-cell leukemia cell line (NALM6) for 96 hours at 1:1 effector:target (E:T) ratio. CD19 CAR T cells were co-cultured with NALM6 at a 2:1 (E:T) ratio. Percentages of live tumor cells on day 0 and day 4 are shown for each co-culture condition. FIG. 3C shows that dual CAR T cells induce specific cytotoxicity against CD19−CD79b+ tumor. Untransduced T, CD19 CAR T and dual CD19−CD79b CAR T were co-cultured with CD19KO B cell lymphoma cell lines (CD19−CD79b+:Daudi -CD19KO and SUDHL6−CD19KO) for 96 hours at a 1:1 effector:target (E:T) ratio, respectively. Percentages of live tumor cells on day 0 and day 4 are shown for each co-culture condition. FIG. 3D shows that dual CAR T cells were not cytotoxic to CD19−CD79b− tumor cells. Untransduced T, CD19 CAR T, and dual CD19−CD79b CAR T cells were co-cultured with CD19−CD79b− K562 tumor cell line for 96 hours at 1:1 effector:target (E:T) ratio. Percentages of live tumor cells on day 0 and day 4 are shown for each co-culture condition. FIG. 3E shows results from experiments in which untransduced T, CD19 CAR T, and dual CD19−CD79b CAR T cells were co-cultured with CD19+CD79b+ Daudi tumor cell line for 24 hours, 48 hours and 96 hours at 1:1 effector:target (E:T) ratio and percent specific lysis was calculated based on absolute number of live tumor cells and normalization to untransduced T cells (% Specific Lysis=1−[live target cells with CAR T cells/live target cells with untransduced T cells). FIG. 3F shows results from experiments in which untransduced T, CD19 CAR T, and dual CD19−CD79b CAR T cells were co-cultured with CD19+CD79b+ SUDHL6 tumor cell line for 24 hours, 48 hours and 96 hours at 1:1 effector:target (E:T) ratio and percent specific lysis was calculated as described for FIG. 3E. FIG. 3G shows results from experiments in which untransduced T, CD19 CAR T, and dual CD19−CD79b CAR T cells were co-cultured with CD19+CD79b− NALM6 tumor cell line for 24 hours, 48 hours and 96 hours at 1:1 effector:target (E:T) ratio and percent specific lysis was calculated as described for FIG. 3E. FIG. 3H shows results from experiments in which untransduced T, CD19 CAR T, and dual CD19−CD79b CAR T cells were co-cultured with CD19−CD79b+Daudi-CD19KO tumor cell line for 24 hours, 48hours and 96 hours at 1:1 effector:target (E:T) ratio and percent specific lysis was calculated as described for FIG. 3E. FIG. 3I shows results from experiments in which untransduced T, CD19 CAR T, and dual CD19−CD79b CAR T cells were co-cultured with CD19−CD79b+SUDHL6−CD19KO tumor cell line for 24 hours, 48 hours and 96 hours at 1:1 effector:target (E:T) ratio and percent specific lysis was calculated as described for FIG. 3E.

FIGS. 4A-4H show results demonstrating that dual CD19−CD79b CAR T cells proliferate in response to B-cell leukemia or lymphoma tumor cells in CD19 or CD79b dependent manner. Untransduced T, CD19 CAR T, and dual CD19−CD79b T cells labeled with Cell Trace™ Far Red were co-cultured with B-cell lymphoma or leukemia tumor cells, Daudi (FIG. 4A), SUDHL6 (FIG. 4B), PDX203 (FIG. 4C), PDX300 (FIG. 4D), NALM6 (FIG. 4E), Daudi-CD19KO (FIG. 4F), SUDHL6−CD19KO (FIG. 4G), and K562 (FIG. 4H) for 96 hours at 1:1 effector:target (E:T) ratio. Percentage of proliferating T cells was determined based on dye dilution on day 4 and indicated in each plot.

FIG. 5A-5B shows results from the design of example dual CD19/CD79b CAR constructs and transfection efficiency in 293T cells. Percentages of 293T cells expressing CD19 or CD79b CAR or both are shown. FIG. 5B shows results from the design of example dual CD19/CD79b CAR constructs and transduction efficiency in primary T cells. Percentages of primary T cells expressing CD19 or CD79b CAR or both are shown.

FIGS. 6A-6C show results demonstrating that CD19−CD79b dual CAR T cells produced effector cytokines in response to B-cell lymphoma and leukemia cell lines in a CD19- or CD79b-dependent manner. Dual CD19−CD79b CAR T cells specifically recognized and released IFN-γ (FIG. 6A), IL-2 (FIG. 6B), and TNF-α (FIG. 6C) in response to B-cell lymphoma cell lines Daudi, SUDHL6, PDX203-5D4, and PDX300-5E6 that are CD19+CD79b+, B-cell leukemia cell line NALM6 that is CD19+CD79b−, and Daudi-CD19KO and SUDHL6−CD19KO lymphoma cell lines that are CD19−CD79b+, but not in response to CD19−CD79b− K562 tumor cells. CD19 CAR T cells were used as a control. In FIGS. 6A and 6B, there is no detectable untransduced (UTD) and in the groupings of two bars the bar on the left is CD19CAR. In FIG. 6C, UTD (where it is present) is the leftmost bar in the groupings of three bars and CD19−CD79b CAR is the rightmost bar in the groupings of three bars.

FIGS. 7A-7C show results demonstrating that CD19−CD79b dual CAR T cells have potent antitumor activity in vivo in a NALM6 CD19+CD79b− cell line xenograft model. NOD.Cg-Prkdc^{scid} IL2rg^{tmlWjl}/SzJ (NSG) mice were injected intravenously with 0.2×10⁶ firefly luciferase-expressing NALM6 tumor cells that are CD19+CD79b−. After 4 days, 5×10⁶ CAR+ cells/mouse of CD19CAR T or CD19−CD79b dual CAR T cells, and corresponding untransduced T cells were injected into tumor-bearing mice via tail vein injection. Tumor burden was measured by bioluminescence imaging at the indicated days (FIG. 7A) and quantified by total flux (n=5 mice per group, significance among treatment groups was determined by Mann-Whitney test. p=ns, **, p<0.01) (FIG. 7B); the CD19 CAR and CD19−CD79b CAR lines are along the x-axis). Kaplan-Meier survival curve for mice treated with untransduced T, CD19 CAR T and CD19−CD79b dual CAR T (n=5 mice per group). Significance among treatment groups was determined by log-rank (Mantel-Cox) test (p=ns, **, p<0.01) (FIG. 7C); the CD19 CAR and CD19−CD79b CAR lines are along the top of the image).

FIG. 8 provides results demonstrating that CD19−CD79b dual CAR T cells have potent antitumor activity in vivo in a patient-derived xenograft model with PDX203-5D4 CD19KO that is CD19−CD79b+. NOD.Cg-Prkdc^{scid}Il2rg^{tm1Wjl}/SzJ (NSG) mice were injected intravenously with 0.2×10⁶ firefly luciferase-expressing PDX203-5D4 CD19KO cells. After 15 days, 3×10⁶ CAR+ cells/mouse of CD19CAR T, CD79b CAR T, or CD19−CD79b dual CAR T cells, and corresponding untransduced T cells were injected via tail vein into tumor-bearing mice. Tumor burden was evaluated by bioluminescence imaging on day 0, day 7, day 14, and day 21.

### DETAILED DESCRIPTION

Along with CD19, CD79b is a pan-B-cell antigen that is expressed at high levels in 95-100% of most B-cell lymphomas including DLBCL, FL, MCL, Burkitt lymphoma, marginal zone lymphoma, and lymphoplasmacytic lymphoma.¹¹⁻¹⁵ Expression of CD79b is also high in hairy cell leukemia but diminished in intensity in chronic lymphocytic leukemia (CLL) compared to normal B cells.¹¹ Accordingly, in certain aspects, the present disclosure provides dual chimeric antigen receptor (CAR) immune cells (e.g., dual CAR T cells) targeting CD19 and CD79b, as well as methods of use thereof in treatment of malignancies that express CD19 and CD79b, including B-cell malignancies such as diffuse large B-cell lymphoma, high-grade B-cell lymphoma, follicular lymphoma, marginal zone lymphoma, mantle cell lymphoma, Burkitt lymphoma, lymphoplasmacytic lymphoma, hairy cell leukemia, and chronic lymphocytic leukemia. Aspects of the present disclosure include polynucleotides encoding an anti-CD19 CAR and an anti-CD79b CAR as well as methods for use of such polynucleotides in generating dual CAR T cells.

### I. Polypeptides

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs: 1-64.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs: 1-15, 31, or 33.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids or nucleotides of SEQ ID NOs: 1-64.

In some embodiments, the polypeptide or protein may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs: 1-15 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 1-15.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS: 1-34 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS: 1-64.

In some embodiments, the peptide, polypeptide, or nucleic acid may comprise, may comprise at least, may comprise at most 1, 2, 3, 4, 5, 6, 7, 8, 9, 10, 11, 12, 13, 14, 15, 16, 17, 18, 19, 20, 21, 22, 23, 24, or 25 (or any derivable range therein) contiguous amino acids or nucleotides of any of SEQ ID NOs: 1-64 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs: 1-64.

In some aspects there is a peptide, polypeptide, or nucleic acid starting at position 1, 2, 3, 4, 5, 6, 7, 8, 9, or 10 of any of SEQ ID NOs: 1-64 and comprising, comprising at least, or comprising at most 2, 3, 4, 5, 6, 7, 8, 9, 10, 11, 12, 13, 14, 15, 16, 17, 18, 19, 20, 21, 22, 23, 24, or 25 (or any derivable range therein) contiguous amino acids or nucleotides of any of SEQ ID NOs: 1-64.

The peptide, polypeptide, or nucleic acid may comprise, may comprise at least, or may comprise at most 1, 2, 3, 4, 5, 6, 7, 8, 9, or 10 (or any derivable range therein) amino acid or nucleotide substitutions relative to SEQ ID NOs: 1-64. The peptide, polypeptide, or nucleic acid may comprise, may comprise at least, or may comprise at most 1, 2, 3, 4, 5, 6, 7, 8, 9, or 10 (or any derivable range therein) amino acid or nucleotide substitutions, and the substitution(s) may be at position 1, 2, 3, 4, 5, 6, 7, 8, 9, 10, 11, 12, 13, 14, 15, 16, 17, 18, 19, 20, 21, 22, 23, 24, and/or 25 relative to SEQ ID NOs: 1-64. The substitution(s) may be at any position(s) of SEQ ID NOs: 1-64. For amino acid substitutions, the substitution may be with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. For nucleic acid substitutions, the substitution may be with a guanine, cytosine, adenine, thymine, uracil, or other nucleotide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Sequences

The amino acid sequence of certain polypeptides, including chimeric antigen receptors and portions, regions, and domains thereof, are provided in Table 1.

TABLE 1

| | SEQ | |
|---|---|---|
| | ID | |
| Name | NO: | Sequence |
| GM-CSFRα | 1 | MLLLVTSLLLCELPHPAFLLIP |
| signal | | |
| peptide | | |
| CD19 scFv | 2 | DIQMTQTTSSLSASLGDRVTISCRASQDISKYLNWYQ |
| (FMC63.3) | | QKPDGTVKLLIYHTSRLHSGVPSRFSGSGSGTDYSLTI |
| light | | SNLEQEDIATYFCQQGNTLPYTFGGGTKLEITG |
| chain | | |
| CD19 scFv | 3 | STSGSGKPGSGEGSTKG |
| (FMC63.3) | | |
| linker | | |
| CD19 scFv | 4 | EVKLQESGPGLVAPSQSLSVTCTVSGVSLPDYGVSWI |
| (FMC63.3) | | RQPPRKGLEWLGVIWGSETTYYNSALKSRLTIIKDNS |
| heavy | | KSQVFLKMNSLQTDDTAIYYCAKHYYYGGSYAMDY |
| chain | | WGQGTSVTVSS |
| CD8α | 5 | TTTPAPRPPTPAPTIASQPLSLRPEACRPAAGGAVHTR |
| hinge | | GLDFACD |
| domain | | |
| CD8α | 6 | IYIWAPLAGTCGVLLLSLVITLYCWV |
| trans- | | |
| membrane | | |
| domain | | |
| 4-1BB | 7 | KRGRKKLLYIFKQPFMRPVQTTQEEDGCSCRFPEEEE |
| costimu- | | GGCEL |
| latory | | |
| domain | | |
| CD3ζ | 8 | RVKFSRSADAPAYQQGQNQLYNELNLGRREEYDVL |
| | | DKRRGRDPEMGGKPRRKNPQEGLYNELQKDKMAEA |
| | | YSEIGMKGERRRGKGHDGLYQGLSTATKDTYDALH |
| | | MQALPPR |
| T2A | 9 | GSGEGRGSLLTCGDVEENPGP |
| CD8α | 10 | MALPVTALLLPLALLLHAARP |
| signal | | |
| peptide | | |
| CD79b | 11 | DIVLTQSPASLAVSLGQRATISCKASQSVDYDGDSYM |
| scFv | | NWYQQKPGQPPKLLIYVASNLESGIPARFSGSGSGTD |
| light | | FTLNIHPVEEEDAATYYCQQSNEDPFTFGSGTKLEIN |
| chain | | |
| CD79b | 12 | GGGGSGGGGSGGGGS |
| scFv | | |
| linker | | |
| CD79b | 13 | EVQLQESGAELVKPGASVKMSCKASGYTFTSYWMH |
| scFv | | WVKQRPGQGLEWIGAIDPSDSYTGYNQKFKGKATLT |
| heavy | | VDTSSSTAYMHLSSLTSEDSAVYFCTRSYYGNSWFA |
| chain | | YWGQGTLVTVSA |
| OX40 | 14 | RRDQRLPPDAHKPPGGGSFRTPIQEEQADAHSTLAKI |
| costimu- | | |
| latory | | |
| domain | | |
| Dual | 15 | MLLLVTSLLLCELPHPAFLLIPDIQMTQTTSSLSASLG |
| CD19/ | | DRVTISCRASQDISKYLNWYQQKPDGTVKLLIYHTSR |
| CD79b | | LHSGVPSRFSGSGSGTDYSLTISNLEQEDIATYFCQQG |
| CAR | | NTLPYTFGGGTKLEITGSTSGSGKPGSGEGSTKGEVK |
| polypep- | | LQESGPGLVAPSQSLSVTCTVSGVSLPDYGVSWIRQP |
| tide | | PRKGLEWLGVIWGSETTYYNSALKSRLTIIKDNSKSQ |
| (in- | | VFLKMNSLQTDDTAIYYCAKHYYYGGSYAMDYWG |
| cluding | | QGTSVTVSSTTTPAPRPPTPAPTIASQPLSLRPEACRP |
| T2A | | AAGGAVHTRGLDFACDIYIWAPLAGTCGVLLLSLVITL |
| peptide) | | YCWVKRGRKKLLYIFKQPFMRPVQTTQEEDGCSCRF |
| | | PEEEEGGCELRVKFSRSADAPAYQQGQNQLYNELNL |
| | | GRREEYDVLDKRRGRDPEMGGKPRRKNPQEGLYNE |
| | | LQKDKMAEAYSEIGMKGERRRGKGHDGLYQGLSTA |
| | | TKDTYDALHMQALPPRGSGEGRGSLLTCGDVEENPG |
| | | PMALPVTALLLPLALLLHAARPDIVLTQSPASLAVSL |
| | | GQRATISCKASQSVDYDGDSYMNWYQQKPGQPPKL |
| | | LIYVASNLESGIPARFSGSGSGTDFTLNIHPVEEEDAA |
| | | TYYCQQSNEDPFTFGSGTKLEINGGGGSGGGGSGGG |
| | | GSEVQLQESGAELVKPGASVKMSCKASGYTFTSYW |
| | | MHWVKQRPGQGLEWIGAIDPSDSYTGYNQKFKGKA |
| | | TLTVDTSSSTAYMHLSSLTSEDSAVYFCTRSYYGNSW |
| | | FAYWGQGTLVTVSATSTTTPAPRPPTPAPTIASQPLSL |
| | | RPEACRPAAGGAVHTRGLDFACDIYIWAPLAGTCGV |
| | | LLLSLVITLYCRRDQRLPPDAHKPPGGGSFRTPIQEEQ |
| | | ADAHSTLAKIRVKFSRSADAPAYQQGQNQLYNELNL |
| | | GRREEYDVLDKRRGRDPEMGGKPRRKNPQEGLYNE |
| | | LQKDKMAEAYSEIGMKGERRRGKGHDGLYQGLSTA |
| | | TKDTYDALHMQALPPR |

Provided below are sequences of certain anti-CD79b antibodies, such as those described in PCT Patent Application Publication WO 2021/222944, which may be useful in generation of anti-CD79B CARs disclosed herein:

Clone T26 VH region:
Nucleotide sequence
(SEQ ID NO: 35)
GAGGTGCAGCTGCAGGAGTCTGGGGCTGAGCTGGTGAAGCCTGGGGCTTCAGTG
AAGATGTCCTGCAAGGCTTCTGGCTACACCTTCACCAGCTACTGGATGCACTGGG
TGAAGCAGAGGCCTGGACCAGGCCTTGAGTGGATCGGAGCAATTGATCCTTCAG
ATAGTTATACTGGCTACAATCAAAAGTTCAAGGGCAAGGCCACATTGACTGTAG
ACACATCCTCCAGCACAGCCTACATGCACCTCAGCAGCCTGACATCTGAGGACTC
TGCGGTCTATTTCTGTACAAGAAGCTACTATGGTAACTCCTGGTTTGCTTACTGGG
GCCAAGGGACTCTGGTCACTGTCTCTGCA (357 nt)
Amino acid sequence
(SEQ ID NO: 36)
EVQLQESGAELVKPGASVKMSCKASGYTFTSYWMHWVKQRPGPGLEWIGAIDPSDS
YTGYNQKFKGKATLTVDTSSSTAYMHLSSLTSEDSAVYFCTRSYYGNSWFAYWGQ
GTLVTVSA (119 aa)
T26 VH CDR1:
(SEQ ID NO: 37)
GYTFTSYW
T26 VH CDR2:
(SEQ ID NO: 38)
IDPSDSYT
T26 VH CDR3:
(SEQ ID NO: 39)
NSWFAYWGQGTLV
Clone T26 VL region:
Nucleotide sequence
(SEQ ID NO: 40)
ACATTGTGCTGACCCAATCTCCAGCTTCTTTGGCTGTGTCTCTAGGGCAGAGGGC
CACCATCTCCTGCAAGGCCAGCCAAAGTGTTGATTATGATGGTGATAGTTATATA
AACTGGTACCAACAGAAACCAGGACAGCCACCCAAACTCCTCATCTATGCTGCA
TCCAATCTAGAATCTGGAATCCCAGCCAGGTTTAGTGCCAGTGGGTCTGGGACAG
ACTTCACCCTCAACATCCATCCTGTGGAGGAGGAGGATGTTGCAGCCTATTACTG
TCAGCAAAGTAATGAGGACCCATTCACGTTCGGCTCGGGGACAAGGTTGGAAAT
AAAAC (330 nt)
Amino acid sequence
(SEQ ID NO: 41)
IVLTQSPASLAVSLGQRATISCKASQSVDYDGDSYINWYQQKPGQPPKLLIYAASNLE
SGIPARFSASGSGTDFTLNIHPVEEEDVAAYYCQQSNEDPFTFGSGTRLEIK (110 aa)
T26 VL CDR1:
(SEQ ID NO: 42)
QSVDYDGDSY
T26 VL CDR2:
(SEQ ID NO: 43)
AAS
T26 VL CDR3:
(SEQ ID NO: 44)
QQSNEDPFT
Clone 5B VH region:
Nucleotide sequence
(SEQ ID NO: 45)
GAGGTGCAGCTGCAGGAGTCTGGGGCTGAGCTGGTGAAGCCTGGGGCTTCAGTG
AAGATGTCCTGCAAGGCTTCTGGCTACACCTTCACCAGCTACTGGATGCACTGGG
TGAAGCAGAGGCCTGGACAAGGCCTTGAGTGGATCGGAGCAATTGATCCTTCAG
ATAGTTATACTGGCTACAATCAAAAGTTCAAGGGCAAGGCCACATTGACTGTAG
ACACATCCTCCAGCACAGCCTACATGCACCTCAGCAGCCTGACATCTGAGGACTC
TGCGGTCTATTTCTGTACAAGAAGCTACTATGGTAACTCCTGGTTTGATTACTGG
GGCCAAGGGACTCTGGTCACTGTCTCTGCA (357 nt)
Amino acid sequence
(SEQ ID NO: 46)
EVQLQESGAELVKPGASVKMSCKASGYTFTSYWMHWVKQRPGQGLEWIGAIDPSD
SYTGYNQKFKGKATLTVDTSSSTAYMHLSSLTSEDSAVYFCTRSYYGNSWFDYWGQ
GTLVTVSA (119 aa)
5B VH CDR1:
(SEQ ID NO: 47)
GYTFTSYW
5B VH CDR2:
(SEQ ID NO: 48)
IDPSDSYT
5B VH CDR3:
(SEQ ID NO: 49)
NSWFDYWGQGTLV
Clone 5B VL region:
Nucleotide sequence
(SEQ ID NO: 50)
GACATTGTGCTGACCCAATCTCCAGCTTCTTTGGCTGTGTCTCTAGGGCAGAGGG
CCACCATCTCCTGCAAGGCCAGCCAAAGTGTTGATTATGAAGGTGATAGTTATAT
GAACTGGTACCAACAGAAACCAGGACAGCCACCCAAACTCCTCATCTATGCTGC
ATCCAATCTAGAATCTGGAATCCCAGCCAGGTTTAGTGGCAGTGGGTCTGGGAC
AGACTTCACCCTCAACATCCATCCTGTGGAGGAGGAGGATGCTGCAACCTATCAC
TGTCAGCAAAGTAATGAGGACCCGTTCACGTTCGGAGGGGGGACCAAGTTGGAA
ATAAAA (333 nt)
Amino acid sequence
(SEQ ID NO: 51)
DIVLTQSPASLAVSLGQRATISCKASQSVDYEGDSYMNWYQQKPGQPPKLLIYAASN
LESGIPARFSGSGSGTDFTLNIHPVEEEDAATYHCQQSNEDPFTFGGGTKLEIK
(111 aa)
5B VL CDR1:
(SEQ ID NO: 52)
QSVDYEGDSY
5B VL CDR2:
(SEQ ID NO: 53)
AAS
5B VL CDR3:
(SEQ ID NO: 54)
QQSNEDPFT
Clone 28B VH region:
Nucleotide sequence
(SEQ ID NO: 55)
GAGGTGCAGCTGCAGGAGTCTGGGGCTGAGCTGGTGAAGCCTGGGGCTTCAGTG
AAGATGTCCTGCAAGGCTTCTGGCTACACCTTCACCAGCTACTGGATGCACTGGG
TGAAGCAGAGGCCTGGACAAGGCCTTGAGTGGATCGGAGCAATTGATCCTTCAG
ATAGTTATACTGGCTACAATCAAAAGTTCAAGGGCAAGGCCACATTGACTGTAG
ACACATCCTCCAGCACAGCCTACATGCACCTCAGCAGCCTGACATCTGAGGACTC
TGCGGTCTATTTCTGTACAAGAAGCTACTATGGTAACTCCTGGTTTGCTTACTGGG
GCCAAGGGACTCTGGTCACTGTCTCTGCA (357 nt)
Amino acid sequence
(SEQ ID NO: 56)
EVQLQESGAELVKPGASVKMSCKASGYTFTSYWMHWVKQRPGQGLEWIGAIDPSD
SYTGYNQKFKGKATLTVDTSSSTAYMHLSSLTSEDSAVYFCTRSYYGNSWFAYWGQ
GTLVTVSA (119 aa)
28B VH CDR1:
(SEQ ID NO: 57)
GYTFTSYW
28B VH CDR2:
(SEQ ID NO: 58)
DPSDSYT
28B VH CDR3:
(SEQ ID NO: 59)
SWFAYWGQGTLV
Clone 28B VL region:
Nucleotide sequence
(SEQ ID NO: 60)
GACATTGTGCTGACCCAATCTCCAGCTTCTTTGGCTGTGTCTCTAGGGCAGAGGG
CCACCATCTCCTGCAAGGCCAGCCAAAGTGTTGATTATGATGGTGATAGTTATAT
GAACTGGTACCAACAGAAACCAGGACAGCCACCCAAACTCCTCATTTATGTTGC
ATCCAATCTAGAATCTGGAATCCCAGCCAGGTTTAGTGGCAGTGGGTCTGGGAC
AGACTTCACCCTCAACATCCATCCTGTGGAGGAGGAGGATGCTGCAACCTATTAC
TGTCAGCAAAGTAATGAGGACCCATTCACGTTCGGCTCGGGGACAAAGTTGGAA
ATAAAC (333 nt)
Amino acid sequence
(SEQ ID NO: 61)
DIVLTQSPASLAVSLGQRATISCKASQSVDYDGDSYMNWYQQKPGQPPKLLIYVASN
LESGIPARFSGSGSGTDFTLNIHPVEEEDAATYYCQQSNEDPFTFGSGTKLEIN
(111 aa)
28B VL CDR1:
(SEQ ID NO: 62)
QSVDYDGDSY
28B VL CDR2:
(SEQ ID NO: 63)
VAS
28B VL CDR3:
(SEQ ID NO: 64)
QQSNEDPFT

### B. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### C. Chimeric Antigen Receptors

In some aspects, disclosed are chimeric antigen receptors (CARs). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric CAR construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

Certain embodiments of the present disclosure concern the use of nucleic acids, including nucleic acids encoding a cancer antigen-specific CAR polypeptide, including in some cases a CAR that has been humanized to reduce immunogenicity (hCAR), comprising at least one intracellular signaling domain, a transmembrane domain, and an extracellular domain comprising one or more signaling motifs. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the CAR nucleic acids may be human genes used to enhance cellular immunotherapy for human patients. In a specific embodiment, the disclosure includes a full-length CAR cDNA or coding region. The antigen binding regions or domain can comprise a fragment of the V_{H} and V_{L} chains of a single-chain variable fragment (scFv) derived from a particular human monoclonal antibody (e.g., an anti-CD19 antibody such as FMC63.3 and/or an anti-CD79b antibody such as those described in PCT Patent Application Publication WO 2021/222944). The fragment can also be any number of different antigen binding domains of a human antigen-specific antibody. In a more specific embodiment, the fragment is a cancer antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof, as it is found that introns stabilize the mRNA. Also, it may be further advantageous to use endogenous or exogenous non-coding regions to stabilize the mRNA.

In some aspects, the antigen-specific binding (e.g., anti-CD19, anti-CD79b), or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In another embodiment, a transmembrane domain is used that is not naturally associated with one of the domains of the CAR. In some instances, the transmembrane domain is selected or modified by amino acid substitution to avoid binding of such domains to the transmembrane domains of the same or different surface membrane proteins to minimize interactions with other members of the receptor complex. The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of; also “from”) the alpha, beta or zeta chain of the T-cell receptor, CD28, DAP12, DAP10, NKG2D, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, and so forth. In some embodiments, a transmembrane domain of the present disclosure is a transmembrane domain from CD8α.

In some embodiments, the CAR nucleic acid comprises a sequence encoding other costimulatory receptors, such as a transmembrane domain and one or more intracellular signaling domains. In addition to a primary T cell activation signal, such as may be initiated by CD3ζ and/or FcεRIγ, an additional stimulatory signal for immune effector cell proliferation and effector function following engagement of the chimeric receptor with the target antigen may be utilized. For example, part or all of a human costimulatory receptor for enhanced activation of cells may be utilized that could help improve in vivo persistence and improve the therapeutic success of the adoptive immunotherapy. Examples include costimulatory domains from molecules such as DAP12, DAP10, NKG2D, CD2, CD28, CD27, 4-1BB, (CD137), OX40, ICOS, (CD278), CD30, HVEM, CD40, LFA-1 (CD11a/CD18), and ICAM-1, although in specific alternative embodiments any one of these listed may be excluded from use in a CAR.

In particular embodiments, specific CAR molecules are encompassed herein. In some cases, the antigen binding domain of the CAR is a scFv, and any scFv that binds to a cancer antigen may be utilized herein. In cases wherein an scFv is utilized in the extracellular domain of the CAR, the variable heavy chain and the variable light chain for the scFv may be in any order in N-terminal to C-terminal direction. For example, the variable heavy chain may be on the N-terminal side of the variable light chain, or vice versa. The scFv and/or ligand that binds the antigen in the CAR may or may not be codon optimized. In particular embodiments, a vector encodes a cancer antigen-specific CAR and also encodes one or more other molecules. For example, a vector may encode both a first CAR (e.g., an anti-CD19 CAR) and a second CAR (e.g., an anti-CD79b CAR).

On the same molecule, the cancer antigen-specific CAR may comprise one or more antigen-specific extracellular domains, a specific hinge, a specific transmembrane domain, one or more specific costimulatory domains, and one or more specific activation signals. When more than one antigen-specific extracellular domain is utilized, such as for targeting two different antigens, there may be a linker between the two antigen-specific extracellular domains. Examples of CARs contemplated herein include, without limitation, CD19-specific (also “anti-CD19”) CARs and CD79b-specific (also “anti-CD79b”) CARs.

In particular embodiments of specific CAR molecules, a CAR may utilize DAP10, DAP12, 4-1BB, NKG2D, or other costimulatory domains (which may be referred to herein as an intracytoplasmic domain). In some cases, CD3zeta is utilized without any costimulatory domains. In particular embodiments of specific CAR molecules, a CAR may utilize any suitable transmembrane domain, such as from DAP12, DAP10, 4-1BB, 2B4, OX40, CD27, NKG2D, CD8, CD28, IL12Rβ1, or IL12Rβ2.

### 1. Signal Peptide

Polypeptides (e.g., CARs) of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some embodiments, a signal peptide directs the nascent protein into the endoplasmic reticulum. This is essential if a receptor is to be glycosylated and anchored in the cell membrane.

In some embodiments, the signal peptide is cleaved after passage of the endoplasmic reticulum (ER), i.e., is a cleavable signal peptide. In some embodiments, a restriction site is at the carboxy end of the signal peptide to facilitate cleavage.

In some aspects, a CAR of the present disclosure comprises a GM-CSFRα signal peptide. In some aspects, the GM-CSFRα signal peptide comprises SEQ ID NO:1. In some aspects, a CAR of the present disclosure comprises a CD8α signal peptide. In some aspects, the CD8α signal peptide comprises SEQ ID NO: 10. In some aspects, an anti-CD19 CAR of the disclosure comprises the GM-CSFRα signal peptide. In some aspects, an anti-CD79b CAR of the disclosure comprises the CD8α signal peptide.

### 2. Antigen Binding Domain

Polypeptides of the present disclosure may comprise one or more antigen binding domains. An “antigen binding domain” describes a region of a polypeptide capable of binding to an antigen under appropriate conditions. In some embodiments, an antigen binding domain is a single-chain variable fragment (scFv) based on one or more antibodies (e.g., anti-CD19 antibodies, anti-CD79b antibodies). In some embodiments, an antigen binding domain comprise a variable heavy (VH) region and a variable light (VL) region, with the VH and VL regions being on the same polypeptide. In some embodiments, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10¹⁰M, 10⁻¹¹M, 10⁻¹²M, or 10⁻¹³M. In some embodiments, the KD of the antigen binding region, such as the variable regions (heavy chain and/or light chain variable region), or of the CDRs may be at least 10⁻⁵M, 10⁶M, 10⁻⁷M, 10⁻⁸M, 10⁻⁹M, 10⁻¹⁰M, 10⁻¹¹M, 10⁻¹²M, or 10⁻¹³M (or any derivable range therein).

Binding affinity, K_{A}, or K_{D} can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some embodiments, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

In some embodiments, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some embodiments, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

In some aspects, an antigen-binding domain of the disclosure is an anti-CD19 binding domain. The anti-CD19 binding domain may comprise a VH and VL from an anti-CD19 antibody (e.g., FMC63.3). In some aspects, the anti-CD19 binding domain comprises a VL having SEQ ID NO:2. In some aspects, the anti-CD19 binding domain comprises a VH having SEQ ID NO:4. In some aspects, the VH and the VL are connected by a linker, for example a linker having SEQ ID NO:3.

In some aspects, an antigen-binding domain of the disclosure is an anti-CD79b binding domain. The anti-CD79b binding domain may comprise a VH and VL from an anti-CD79b antibody (e.g., an anti-CD79b antibody described in PCT Patent Application Publication WO 2021/222944, incorporated herein by reference). In some aspects, the anti-CD79b binding domain comprises a VL having SEQ ID NO:11. In some aspects, the anti-CD79b binding domain comprises a VH having SEQ ID NO:13. In some aspects, the VL and the VH are connected by a linker, for example a linker having SEQ ID NO:12.

### 3. Peptide Spacer

A peptide spacer, such as an extracellular spacer may link an antigen-binding domain to a transmembrane domain. In some embodiments, a peptide spacer is flexible enough to allow the antigen-binding domain to orient in different directions to facilitate antigen binding. In one embodiment, the spacer comprises the hinge region from IgG. In some embodiments, the spacer comprises or further comprises the CH2CH3 region of immunoglobulin and portions of CD3. In some embodiments, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some embodiments, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgGl) is generally defined as stretching from Glu216 to Pro230 of human IgGl (Burton (1985) Molec. Immunol., 22:161-206). Hinge regions of other IgG isotypes may be aligned with the IgGl sequence by placing the first and last cysteine residues forming inter-heavy chain disulfide (S-S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some embodiments, the extracellular spacer consists of or comprises a hinge region from an immunoglobulin (e.g., IgG). Immunoglobulin hinge region amino acid sequences are known in the art; see, e.g., Tan et al. (1990) Proc. Natl. Acad. Sci. USA 87:162; and Huck et al. (1986) Nucl. Acids Res.

The length of an extracellular spacer may have effects on the CAR's signaling activity and/or the CAR-T cells' expansion properties in response to antigen-stimulated CAR signaling. In some embodiments, a shorter spacer such as less than 50, 45, 40, 30, 35, 30, 25, 20, 15, 14, 13, 12, 11, or 10 amino acids is used. In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some embodiments, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

In some aspects, a CAR of the present disclosure comprises a CD8α hinge. In some aspects, the hinge comprises SEQ ID NO:5.

### 4. Transmembrane Domain

Polypeptides of the present disclosure may comprise a transmembrane domain. In some embodiments, a transmembrane domain is a hydrophobic alpha helix that spans the membrane. Different transmembrane domains may result in different receptor stability.

In some embodiments, the transmembrane domain is interposed between the extracellular spacer and the cytoplasmic region. In some embodiments, the transmembrane domain is interposed between the extracellular spacer and one or more costimulatory regions. In some embodiments, a linker is between the transmembrane domain and the one or more costimulatory regions.

Any transmembrane domain that provides for insertion of a polypeptide into the cell membrane of a eukaryotic (e.g., mammalian) cell may be suitable for use. In some embodiments, the transmembrane domain is derived from CD28, CD8, CD4, CD3-zeta, CD134, or CD7.

In some aspects, a CAR of the present disclosure comprises a CD8α transmembrane domain. In some aspects, the CD8α transmembrane domain comprises SEQ ID NO:6.

### 5. Cytoplasmic Region

After antigen recognition, receptors of the present disclosure may cluster and a signal transmitted to the cell through the cytoplasmic region. In some embodiments, the costimulatory domains described herein are part of the cytoplasmic region. In some embodiments, the cytoplasmic region comprises an intracellular signaling domain. An intracellular signaling domain may comprise a primary signaling domain and one or more costimulatory domains.

Cytoplasmic regions and/or costimulatiory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes at least one (e.g., one, two, three, four, five, six, etc.) ITAM motif as described herein. In some embodiments, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an ΓΓAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

In some embodiments, a suitable cytoplasmic region can comprise an ITAM motif-containing portion of the full length DAP12 amino acid sequence. In some embodiments, the cytoplasmic region is derived from FCER1G (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). In some embodiments, a suitable cytoplasmic region can comprise an ITAM motif-containing portion of the full length FCER1G amino acid sequence.

In some embodiments, the cytoplasmic region is derived from T cell surface glycoprotein CD3 delta chain (also known as CD3D; CD3-DELTA; T3D; CD3 antigen, delta subunit; CD3 delta; CD3δ; CD3d antigen, delta polypeptide (TiT3 complex); OKT3, delta chain; T cell receptor T3 delta chain; T cell surface glycoprotein CD3 delta chain; etc.). In some embodiments, a suitable cytoplasmic region can comprise an ITAM motif-containing portion of the full length CD3 delta amino acid sequence. In some embodiments, the cytoplasmic region is derived from T cell surface glycoprotein CD3 epsilon chain (also known as CD3e, CD3ε; T cell surface antigen T3/Leu-4 epsilon chain, T cell surface glycoprotein CD3 epsilon chain, AI504783, CD3, CD3-epsilon, T3e, etc.). In some embodiments, a suitable cytoplasmic region can comprise an ITAM motif-containing portion of the full length CD3 epsilon amino acid sequence. In some embodiments, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). In some embodiments, a suitable cytoplasmic region can comprise an ITAM motif-containing portion of the full length CD3 gamma amino acid sequence. In some embodiments, the cytoplasmic region is derived from T cell surface glycoprotein CD3 zeta chain (also known as CD3Z, CD3ζ, T cell receptor T3 zeta chain, CD247, CD3-ZETA, CD3H, CD3Q, T3Z, TCRZ, etc.). In some embodiments, a suitable cytoplasmic region can comprise an ITAM motif-containing portion of the full length CD3 zeta amino acid sequence.

### 6. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some embodiments, the costimulatory region is derived from an intracellular portion of the transmembrane protein 4-1BB (also known as TNFRSF9; CD137; CDw137; ILA; etc.). In some embodiments, the costimulatory region is derived from an intracellular portion of the transmembrane protein CD28 (also known as Tp44). In some embodiments, the costimulatory region is derived from an intracellular portion of the transmembrane protein ICOS (also known as AILIM, CD278, and CVID1). In some embodiments, the costimulatory region is derived from an intracellular portion of the transmembrane protein OX-40 (also known as TNFRSF4, RP5-902P8.3, ACT35, CD134, OX40, TXGP1L). In some embodiments, the costimulatory region is derived from an intracellular portion of the transmembrane protein BTLA (also known as BTLA1 and CD272). In some embodiments, the costimulatory region is derived from an intracellular portion of the transmembrane protein CD27 (also known as S 152, T14, TNFRSF7, and Tp55). In some embodiments, the costimulatory region is derived from an intracellular portion of the transmembrane protein CD30 (also known as TNFRSF8, D1S166E, and Ki-1). In some embodiments, the costimulatory region is derived from an intracellular portion of the transmembrane protein GITR (also known as TNFRSF18, RP5-902P8.2, AITR, CD357, and GITR-D). In some embodiments, the costimulatory region derived from an intracellular portion of the transmembrane protein HVEM (also known as TNFRSF14, RP3-395M20.6, ATAR, CD270, HVEA, HVEM, LIGHTR, and TR2).

In some aspects, a CAR of the present disclosure comprises a 4-1BB costimulatory domain. In some aspects, the 4-1BB costimulatory domain comprises SEQ ID NO:7. In some aspects, a CAR of the present disclosure comprises an OX40 costimulatory domain. In some aspects, the OX40 costimulatory domain comprises SEQ ID NO:14. In some aspects, an anti-CD19 CAR of the disclosure comprises the 4-1BB costimulatory domain. In some aspects, an anti-CD79b CAR of the disclosure comprises the OX40 costimulatory domain.

### 7. Peptide Linkers

In some embodiments, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

### II. Nucleic Acids

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Vectors

The polypeptides of the disclosure, including dual CARs of the disclosure, may be delivered to recipient immune cells by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the immune cell is transduced with a vector encoding two antigen-targeting CARs cell, the CARs may or may not be comprised on or with the same vector. In some cases, the CARs are expressed from the same vector molecule, such as the same viral vector molecule. In such cases, the expression of the CARs may or may not be regulated by the same regulatory element(s). When the CARs are on the same vector, they may or may not be expressed as separate polypeptides. In cases wherein they are expressed as separate polypeptides, they may be separated on the vector by a 2A element or IRES element (or both kinds may be used on the same vector once or more than once), for example. In some aspects, a dual CAR expressing vector of the disclosure is a multicistronic (e.g., bicistronic) vector expressing both an anti-CD19 CAR and an anti-CD79b CAR separated by a 2A element. In some aspects, the 2A element is a T2A element. In some aspects, the 2A element is a P2A element. In some aspects, the 2A element is an E2A element.

### B. Sequences

Certain nucleotide sequences of the polynucleotides, including polynucleotides expressing chimeric antigen receptors and portions and regions thereof, are provided in Table 2.

TABLE 2

| | SEQ | |
|---|---|---|
| | ID | |
| Name | NO: | Sequence |
| GM-CSFRα signal peptide | 16 | ATGCTGCTGCTCGTGACCTCCCTGCTGCTGTGCGAG |
| | | CTGCCACACCCTGCCTTCCTGCTGATCCCT |
| CD19 scFv (FMC63.3) light | 17 | GACATCCAGATGACCCAGACCACAAGCTCCCTGTC |
| chain | | CGCCTCTCTGGGCGACAGAGTGACAATCTCTTGTA |
| | | GGGCCAGCCAGGATATCTCCAAGTATCTGAACTGG |
| | | TACCAGCAGAAGCCAGATGGCACCGTGAAGCTGCT |
| | | GATCTATCACACATCTAGGCTGCACAGCGGAGTGC |
| | | CATCCCGGTTTAGCGGATCCGGATCTGGAACCGAC |
| | | TACTCTCTGACAATCAGCAACCTGGAGCAGGAGGA |
| | | TATCGCCACCTATTTCTGCCAGCAGGGCAATACCCT |
| | | GCCTTACACATTTGGCGGCGGCACAAAGCTGGAGA |
| | | TCACCGGC |
| CD19 scFv (FMC63.3) | 18 | AGCACATCCGGATCTGGCAAGCCAGGATCCGGAGA |
| linker | | GGGATCTACCAAGGGA |
| CD19 scFv (FMC63.3) heavy | 19 | GAGGTGAAGCTGCAGGAGAGCGGACCAGGACTGG |
| chain | | TGGCACCCAGCCAGTCCCTGTCTGTGACCTGTACA |
| | | GTGTCCGGCGTGTCTCTGCCAGACTACGGCGTGAG |
| | | CTGGATCAGGCAGCCACCTAGGAAGGGACTGGAGT |
| | | GGCTGGGCGTGATCTGGGGCTCCGAGACCACATAC |
| | | TATAATAGCGCCCTGAAGTCCAGACTGACCATCAT |
| | | CAAGGATAACAGCAAGTCCCAGGTGTTCCTGAAGA |
| | | TGAATTCCCTGCAGACCGACGATACAGCCATCTAC |
| | | TATTGCGCCAAGCACTACTATTACGGCGGCTCCTAT |
| | | GCCATGGACTACTGGGGCCAGGGCACCTCTGTGAC |
| | | AGTGTCTAGC |
| CD8α hinge domain | 20 | ACCACGACACCGGCTCCAAGACCACCCACTCCCGC |
| | | GCCCACAATCGCTTCCCAACCATTGAGCCTTAGGC |
| | | CTGAGGCGTGCCGACCGGCAGCGGGTGGAGCTGTA |
| | | CACACTAGGGGGCTCGACTTTGCGTGTGAC |
| CD8α transmembrane | 21 | ATTTATATCTGGGCGCCATTGGCCGGTACTTGTGGT |
| domain | | GTATTGCTCCTCTCCCTTGTTATTACATTGTATTGCT |
| | | GGGTG |
| 4-1BB costimulatory | 22 | AAACGAGGTAGAAAAAAACTTCTTTATATATTCAA |
| domain | | ACAACCATTTATGAGACCAGTACAAACTACTCAAG |
| | | AGGAAGATGGATGTAGTTGTCGATTTCCAGAAGAA |
| | | GAAGAAGGAGGATGTGAACTG |
| CD3ζ | 23 | AGGGTAAAATTTAGTAGATCCGCCGACGCCCCGGC |
| | | TTACCAGCAGGGTCAGAATCAACTCTATAACGAGC |
| | | TGAACCTCGGGCGCAGAGAAGAGTACGACGTCTTG |
| | | GATAAGCGGAGAGGGCGAGACCCTGAAATGGGGG |
| | | GAAAACCGCGACGGAAGAATCCTCAAGAGGGACT |
| | | CTACAACGAGTTGCAGAAGGACAAAATGGCGGAA |
| | | GCGTACAGTGAGATAGGAATGAAAGGAGAACGCA |
| | | GACGCGGTAAGGGCCATGACGGGCTCTACCAGGGC |
| | | CTGTCAACAGCTACAAAAGATACATACGACGCCCT |
| | | TCATATGCAGGCGCTGCCACCACGG |
| T2A | 24 | GGTTCTGGAGAGGGAAGGGGCTCTCTGCTGACATG |
| | | CGGCGATGTGGAGGAGAATCCTGGCCCA |
| CD8α signal peptide | 25 | ATGGCCCTGCCTGTGACAGCCCTGCTGCTGCCTCTG |
| | | GCTCTGCTGCTGCATGCCGCTAGACCC |
| CD79b scFv light chain | 26 | GACATTGTGCTGACCCAATCTCCAGCTTCTTTGGCT |
| | | GTGTCTCTAGGGCAGAGGGCCACCATCTCCTGCAA |
| | | GGCCAGCCAAAGTGTTGATTATGATGGTGATAGTT |
| | | ATATGAACTGGTACCAACAGAAACCAGGACAGCC |
| | | ACCCAAACTCCTCATTTATGTTGCATCCAATCTAGA |
| | | ATCTGGAATCCCAGCCAGGTTTAGTGGCAGTGGGT |
| | | CTGGGACAGACTTCACCCTCAACATCCATCCTGTG |
| | | GAGGAGGAGGATGCTGCAACCTATTACTGTCAGCA |
| | | AAGTAATGAGGACCCATTCACGTTCGGCTCGGGGA |
| | | CAAAGTTGGAAATAAAC |
| CD79b scFv linker | 27 | GGAGGAGGTGGTAGTGGTGGAGGAGGAAGTGGAG |
| | | GAGGAGGAAGT |
| CD79b scFv heavy chain | 28 | GAGGTGCAGCTGCAGGAGTCTGGGGCTGAGCTGGT |
| | | GAAGCCTGGGGCTTCAGTGAAGATGTCCTGCAAGG |
| | | CTTCTGGCTACACCTTCACCAGCTACTGGATGCACT |
| | | GGGTGAAGCAGAGGCCTGGACAAGGCCTTGAGTG |
| | | GATCGGAGCAATTGATCCTTCAGATAGTTATACTG |
| | | GCTACAATCAAAAGTTCAAGGGCAAGGCCACATTG |
| | | ACTGTAGACACATCCTCCAGCACAGCCTACATGCA |
| | | CCTCAGCAGCCTGACATCTGAGGACTCTGCGGTCT |
| | | ATTTCTGTACAAGAAGCTACTATGGTAACTCCTGGT |
| | | TTGCTTACTGGGGCCAAGGGACTCTGGTCACTGTCT |
| | | CTGCA |
| OX40 costimulatory domain | 29 | CGGAGGGACCAGAGGCTGCCCCCCGATGCCCACAA |
| | | GCCCCCTGGGGGAGGCAGTTTCCGGACCCCCATCC |
| | | AAGAGGAGCAGGCCGACGCCCACTCCACCCTGGCC |
| | | AAGATC |
| Dual CD19/CD79b CAR | 30 | ATGCTGCTGCTCGTGACCTCCCTGCTGCTGTGCGAG |
| expressing polynucleotide | | CTGCCACACCCTGCCTTCCTGCTGATCCCTGACATC |
| (including T2A element) | | CAGATGACCCAGACCACAAGCTCCCTGTCCGCCTC |
| | | TCTGGGCGACAGAGTGACAATCTCTTGTAGGGCCA |
| | | GCCAGGATATCTCCAAGTATCTGAACTGGTACCAG |
| | | CAGAAGCCAGATGGCACCGTGAAGCTGCTGATCTA |
| | | TCACACATCTAGGCTGCACAGCGGAGTGCCATCCC |
| | | GGTTTAGCGGATCCGGATCTGGAACCGACTACTCT |
| | | CTGACAATCAGCAACCTGGAGCAGGAGGATATCGC |
| | | CACCTATTTCTGCCAGCAGGGCAATACCCTGCCTTA |
| | | CACATTTGGCGGCGGCACAAAGCTGGAGATCACCG |
| | | GCAGCACATCCGGATCTGGCAAGCCAGGATCCGGA |
| | | GAGGGATCTACCAAGGGAGAGGTGAAGCTGCAGG |
| | | AGAGCGGACCAGGACTGGTGGCACCCAGCCAGTCC |
| | | CTGTCTGTGACCTGTACAGTGTCCGGCGTGTCTCTG |
| | | CCAGACTACGGCGTGAGCTGGATCAGGCAGCCACC |
| | | TAGGAAGGGACTGGAGTGGCTGGGCGTGATCTGGG |
| | | GCTCCGAGACCACATACTATAATAGCGCCCTGAAG |
| | | TCCAGACTGACCATCATCAAGGATAACAGCAAGTC |
| | | CCAGGTGTTCCTGAAGATGAATTCCCTGCAGACCG |
| | | ACGATACAGCCATCTACTATTGCGCCAAGCACTAC |
| | | TATTACGGCGGCTCCTATGCCATGGACTACTGGGG |
| | | CCAGGGCACCTCTGTGACAGTGTCTAGCACCACGA |
| | | CACCGGCTCCAAGACCACCCACTCCCGCGCCCACA |
| | | ATCGCTTCCCAACCATTGAGCCTTAGGCCTGAGGC |
| | | GTGCCGACCGGCAGCGGGTGGAGCTGTACACACTA |
| | | GGGGGCTCGACTTTGCGTGTGACATTTATATCTGG |
| | | GCGCCATTGGCCGGTACTTGTGGTGTATTGCTCCTC |
| | | TCCCTTGTTATTACATTGTATTGCTGGGTGAAACGA |
| | | GGTAGAAAAAAACTTCTTTATATATTCAAACAACC |
| | | ATTTATGAGACCAGTACAAACTACTCAAGAGGAAG |
| | | ATGGATGTAGTTGTCGATTTCCAGAAGAAGAAGAA |
| | | GGAGGATGTGAACTGAGGGTAAAATTTAGTAGATC |
| | | CGCCGACGCCCCGGCTTACCAGCAGGGTCAGAATC |
| | | AACTCTATAACGAGCTGAACCTCGGGCGCAGAGAA |
| | | GAGTACGACGTCTTGGATAAGCGGAGAGGGCGAG |
| | | ACCCTGAAATGGGGGGAAAACCGCGACGGAAGAA |
| | | TCCTCAAGAGGGACTCTACAACGAGTTGCAGAAGG |
| | | ACAAAATGGCGGAAGCGTACAGTGAGATAGGAAT |
| | | GAAAGGAGAACGCAGACGCGGTAAGGGCCATGAC |
| | | GGGCTCTACCAGGGCCTGTCAACAGCTACAAAAGA |
| | | TACATACGACGCCCTTCATATGCAGGCGCTGCCAC |
| | | CACGGGGTTCTGGAGAGGGAAGGGGCTCTCTGCTG |
| | | ACATGCGGCGATGTGGAGGAGAATCCTGGCCCAAT |
| | | GGCCCTGCCTGTGACAGCCCTGCTGCTGCCTCTGGC |
| | | TCTGCTGCTGCATGCCGCTAGACCCGACATTGTGCT |
| | | GACCCAATCTCCAGCTTCTTTGGCTGTGTCTCTAGG |
| | | GCAGAGGGCCACCATCTCCTGCAAGGCCAGCCAAA |
| | | GTGTTGATTATGATGGTGATAGTTATATGAACTGGT |
| | | ACCAACAGAAACCAGGACAGCCACCCAAACTCCTC |
| | | ATTTATGTTGCATCCAATCTAGAATCTGGAATCCCA |
| | | GCCAGGTTTAGTGGCAGTGGGTCTGGGACAGACTT |
| | | CACCCTCAACATCCATCCTGTGGAGGAGGAGGATG |
| | | CTGCAACCTATTACTGTCAGCAAAGTAATGAGGAC |
| | | CCATTCACGTTCGGCTCGGGGACAAAGTTGGAAAT |
| | | AAACGGAGGAGGTGGTAGTGGTGGAGGAGGAAGT |
| | | GGAGGAGGAGGAAGTGAGGTGCAGCTGCAGGAGT |
| | | CTGGGGCTGAGCTGGTGAAGCCTGGGGCTTCAGTG |
| | | AAGATGTCCTGCAAGGCTTCTGGCTACACCTTCAC |
| | | CAGCTACTGGATGCACTGGGTGAAGCAGAGGCCTG |
| | | GACAAGGCCTTGAGTGGATCGGAGCAATTGATCCT |
| | | TCAGATAGTTATACTGGCTACAATCAAAAGTTCAA |
| | | GGGCAAGGCCACATTGACTGTAGACACATCCTCCA |
| | | GCACAGCCTACATGCACCTCAGCAGCCTGACATCT |
| | | GAGGACTCTGCGGTCTATTTCTGTACAAGAAGCTA |
| | | CTATGGTAACTCCTGGTTTGCTTACTGGGGCCAAG |
| | | GGACTCTGGTCACTGTCTCTGCAACTAGTACAACT |
| | | ACTCCAGCACCACGACCACCAACACCTGCTCCAAC |
| | | TATCGCATCTCAACCACTTTCTCTACGTCCAGAAGC |
| | | ATGCCGACCAGCTGCAGGAGGTGCAGTTCATACGA |
| | | GAGGTCTAGATTTCGCATGTGATATCTACATCTGG |
| | | GCACCATTGGCTGGGACTTGTGGTGTCCTTCTCCTA |
| | | TCACTGGTTATCACCCTTTACTGCCGGAGGGACCA |
| | | GAGGCTGCCCCCCGATGCCCACAAGCCCCCTGGGG |
| | | GAGGCAGTTTCCGGACCCCCATCCAAGAGGAGCAG |
| | | GCCGACGCCCACTCCACCCTGGCCAAGATCAGGGT |
| | | GAAGTTTTCTCGCAGCGCCGATGCCCCAGCCTATCA |
| | | GCAGGGCCAGAACCAGCTGTACAACGAGCTGAATC |
| | | TGGGCAGGCGCGAGGAGTACGACGTGCTGGATAA |
| | | GAGGAGAGGAAGGGATCCAGAGATGGGAGGCAAG |
| | | CCTAGGCGCAAGAACCCACAGGAGGGCCTGTATAA |
| | | TGAGCTGCAGAAGGACAAGATGGCCGAGGCCTAC |
| | | AGCGAGATCGGCATGAAGGGAGAGAGGAGAAGGG |
| | | GCAAGGGACACGATGGCCTGTATCAGGGCCTGTCC |
| | | ACAGCCACCAAGGACACCTACGATGCACTGCACAT |
| | | GCAGGCACTGCCACCTAGATAA |

### III. Cancer Therapy

In some embodiments, methods of the disclosure comprise administering a cancer therapy to a subject. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the present disclosure provides methods for immunotherapy comprising administering an effective amount of the compositions that comprise the CAR(s), of the present disclosure. In one embodiment, a medical disease or disorder is treated by administration of a dual CAR-expressing cell population that elicits an immune response. In certain embodiments of the present disclosure, cancer is treated by administration of a dual CAR immune cell population that elicits an immune response. Provided herein are methods for treating or delaying progression of cancer in an individual comprising administering to the individual an effective amount of an antigen-specific cell therapy. The present methods may be applied for the treatment of immune disorders, solid cancers, and hematologic cancers, as examples. Specifically, the cancer may be a B cell malignancy, such as diffuse large B-cell lymphoma, high-grade B-cell lymphoma, follicular lymphoma, marginal zone lymphoma, mantle cell lymphoma, Burkitt lymphoma, lymphoplasmacytic lymphoma, hairy cell leukemia, and chronic lymphocytic leukemia.

Certain embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In some embodiments of the methods of the present disclosure, activated CD4 and/or CD8 T cells in the individual are characterized by γ-IFN producing CD4 and/or CD8 T cells and/or enhanced cytolytic activity relative to prior to the administration of the combination. γ-IFN may be measured by any means known in the art, including, e.g., intracellular cytokine staining (ICS) involving cell fixation, permeabilization, and staining with an antibody against γ-IFN. Cytolytic activity may be measured by any means known in the art, e.g., using a cell killing assay with mixed effector and target cells.

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T cell growth factor that promotes the growth and activation of the autologous T cells is administered to the subject either concomitantly with the autologous T cells or subsequently to the autologous T cells. The T cell growth factor can be any suitable growth factor that promotes the growth and activation of the autologous T cells. Examples of suitable T-cell growth factors include interleukin (IL)-2, IL-7, IL-15, and/or IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

The T cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of T cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ T cells/m². In a certain embodiment, the dose used in the treatment of human subjects ranges from about 3.8×10⁹ to about 3.8×10¹⁰ T cells/m². In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of T cells is readily determined by one of skill in the art based on the age, weight, sex, and physiological condition of the subject. Effective doses can be extrapolated from dose-response curves derived from in vitro or animal model test systems.

In certain embodiments of the present disclosure, an effective amount of dual CAR-expressing immune cells are delivered to an individual in need thereof, such as an individual that has cancer. The cells then enhance the individual's immune system to attack the cancer cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

In specific embodiments, the cells that have been engineered to express a dual CAR are provided to an individual in a therapeutically effective amount (in a range from 10³ to 10¹⁰) that ameliorates at least one symptom related to cancer cells in the individual. A therapeutically effective amount may be from 10³ to 10¹⁰, 10³ to 10⁹, 10³ to 10⁸, 10³ to 10⁷, 10³ to 10⁶, 10³ to 10⁵, 10³ to 10⁴, 10⁴ to 10¹⁰, 10⁴ to 10⁹, 10⁴ to 10⁸, 10⁴ to 10⁷, 10⁴ to 10⁶, 10⁴ to 10⁵, 10⁵ to 10¹⁰, 10⁵ to 10⁹, 10⁵ to 10⁸, 10⁵ to 10⁷, 10⁵ to 10⁶, 10⁶ to 10¹⁰, 10⁶ to 10⁹, 10⁶ to 10⁸, 10⁶ to 10⁷, 10⁷ to 10¹⁰, 10⁷ to 10⁹, 10⁷ to 10⁸, 10⁸ to 10¹⁰, 10⁸ to 10^{9, or} 10⁹ to 10¹⁰ cells. Thus, in particular embodiments an individual having a certain cancer is provided once or multiple times a therapeutically effective amount of cells expressing dual CARs.

### IV. Infinite Immune Cells

Certain embodiments of the present disclosure concern immune cells that are engineered to express one or more genes. The expression of the one or more genes directly or indirectly results in the increased lifespan of the cells compared to cells that lack the expression of the one or more genes. In particular embodiments, the cells are manipulated to express the one or more genes, including one or more heterologous genes. In other cases, the cells are manipulated to have upregulation of expression of the one or more genes that are endogenous to the cells, such as through manipulation of one or more regulatory elements of the one or more endogenous genes to the cells.

In particular embodiments, immune cells are manipulated to express BCL6 and one or more pro-survival genes or anti-apoptotic genes or cell survival-promoting genes (and there may or may not be overlap in a gene that is classified as pro-survivial or anti-apoptotic or cell survival-promoting). As used herein, the pro-survival gene refers to a nucleic acid polymer that can exert anti-apoptosis function or promote survival by any mechanism. The nucleic acid polymer that can exert anti-apoptosis function may be one or more of Bc12 family genes such as BCL-xL, BCL-2, MCL-1, Bcl-w, Bfl-1, BCL-B, etc. The nucleic acid polymer that can exert anti-apoptosis function may be one or more of inhibitor of apoptosis (IAP) family genes, such as XIAP, c-IAP1, C-IAP2, NAIP, and Survivin, etc. The nucleic acid polymer that can exert anti-apoptosis function may be able to inhibit or knock out expression of one or more caspases that play a role in apoptosis, such as Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14. Nucleic acid polymers for knockdown or knock-out could be an shRNA expression cassette, or these caspase genes can also be knocked out by gene editing method (CRISPR, TALEN, Zinc finger method, etc.). The nucleic acid polymer that can exert anti-apoptosis function may be able to inhibit or knock out expression of one or more pro-apoptotic genes, such as BIM, Puma, Noxa, Bik, Bmf, Bad, Hrk, Bid, BAX, BAK, BOK, etc. The nucleic acid polymer that can exert anti-apoptosis function may have an anti-apoptotic effect, such as insulin-like growth factor (IGF-1), Hsp70, Hsp27, cFLIP, BNIP3, FADD, Akt, and NF-κB, Raf-1 and MEK1, p90Rsk, C-Jun, BNIP2, BAG1, HSPA9, HSP90B1, miRNA21, miR-106b-25, miR-206, miR-221/222, miR-17-92, miR-133, miR-143, miR-145, miR-155, miR-330, etc.

Infinite T cells may be generated with either wild type or mutant BCL6. The inventors determined that infinite T cells could be generated with either wildtype BCL6 or mutant BCL6 with a single particular nucleotide difference—the codon of the amino acid at position 395 in wild type BCL6 is CCT (encoding Proline/P) and the codon of the amino acid at position 395 in mutant BCL6 is CTT (encoding Leucine/L). The nucleotide and amino acid sequences for the two BCL6 genes are shown below (with the point of mutation in the wildtype sequence being underlined).

The aa sequence of wildtype BCL6:

(SEQ ID NO: 31)
MASPADSCIQFTRHASDVLLNLNRLRSRDILTDVVIVVSREQFRAHKTVL
MACSGLFYSIFTDQLKCNLSVINLDPEINPEGFCILLDFMYTSRLNLREG
NIMAVMATAMYLQMEHVVDTCRKFIKASEAEMVSAIKPPREEFLNSRMLM
PQDIMAYRGREVVENNLPLRSAPGCESRAFAPSLYSGLSTPPASYSMYSH
LPVSSLLFSDEEFRDVRMPVANPFPKERALPCDSARPVPGEYSRPTLEVS
PNVCHSNIYSPKETIPEEARSDMHYSVAEGLKPAAPSARNAPYFPCDKAS
KEEERPSSEDEIALHFEPPNAPLNRKGLVSPQSPQKSDCQPNSPTESCSS
KNACILQASGSPPAKSPTDPKACNWKKYKFIVLNSLNQNAKPEGPEQAEL
GRLSPRAYTAPPACQPPMEPENLDLQSPTKLSASGEDSTIPQASRLNNIV
NRSMTGSPRSSSESHSPLYMHPPKCTSCGSQSPQHAEMCLHTAGPTFPEE
MGETQSEYSDSSCENGAFFCNECDCRFSEEASLKRHTLQTHSDKPYKCDR
CQASFRYKGNLASHKTVHTGEKPYRCNICGAQFNRPANLKTHTRIHSGEK
PYKCETCGARFVQVAHLRAHVLIHTGEKPYPCEICGTRFRHLQTLKSHLR
IHTGEKPYHCEKCNLHFRHKSQLRLHLRQKHGAITNTKVQYRVSATDLPP
ELPKAC

The nucleotide sequence of wildtype BCL6 (with the codon for the point of mutation in the wildtype sequence being underlined):

(SEQ ID NO: 32)
ATGgcctcgccggctgacagctgtatccagttcacccgccatgc
cagtgatgttcttctcaaccttaatgtctccggagtcgagacat
cttgactgatgttgtcattgttgtgagccgtgagcagtttagag
cccataaaacggtcctcatggcctgcagtggcctgttctatagc
atctttacagaccagttgaaatgcaaccttagtgtgatcaatct
agatcctgagatcaaccctgagggattctgcatcctcctggact
tcatgtacacatctcggctcaatttgcgggagggcaacatcatg
gctgtgatggccacggctatgtacctgcagatggagcatgttgt
ggacacttgccggaagtttattaaggccagtgaagcagagatgg
tttctgccatcaagcctcctcgtgaagagttcctcaacagccgg
atgctgatgccccaagacatcatggcctatcggggtcgtgaggt
ggtggagaacaacctgccactgaggagcgcccctgggtgtgaga
gcagagcctttgcccccagcctgtacagtggcctgtccacaccg
ccagcctcttattccatgtacagccacctccctgtcagcagcct
cctcttctccgatgaggagtttcgggatgtccggatgcctgtgg
ccaaccccttccccaaggagcgggcactcccatgtgatagtgcc
aggccagtccctggtgagtacagccggccgactttggaggtgtc
ccccaatgtgtgccacagcaatatctattcacccaaggaaacaa
tcccagaagaggcacgaagtgatatgcactacagtgtggctgag
ggcctcaaacctgctgccccctcagcccgaaatgccccctactt
cccttgtgacaaggccagcaaagaagaagagagaccctcctcgg
aagatgagattgccctgcatttcgagccccccaatgcacccctg
aaccggaagggtctggttagtccacagagcccccagaaatctga
ctgccagcccaactcgcccacagagtcctgcagcagtaagaatg
cctgcatcctccaggcttctggctcccctccagccaagagcccc
actgaccccaaagcctgcaactggaagaaatacaagttcatcgt
gctcaacagcctcaaccagaatgccaaaccagaggggcCtgagc
aggctgagctgggccgcctttccccacgagcctacacggcccca
cctgcctgccagccacccatggagcctgagaaccttgacctcca
gtccccaaccaagctgagtgccagcggggaggactccaccatcc
cacaagccagccggctcaataacatcgttaacaggtccatgacg
ggctctccccgcagcagcagcgagagccactcaccactctacat
gcaccccccgaagtgcacgtcctgcggctctcagtccccacagc
atgcagagatgtgcctccacaccgctggccccacgttccctgag
gagatgggagagacccagtctgagtactcagattctagctgtga
gaacggggccttcttctgcaatgagtgtgactgccgcttctctg
aggaggcctcactcaagaggcacacgctgcagacccacagtgac
aaaccctacaagtgtgaccgctgccaggcctccttccgctacaa
gggcaacctcgccagccacaagaccgtccataccggtgagaaac
cctatcgttgcaacatctgtggggcccagttcaaccggccagcc
aacctgaaaacccacactcgaattcactctggagagaagcccta
caaatgcgaaacctgcggagccagatttgtacaggtggcccacc
tccgtgcccatgtgcttatccacactggtgagaagccctatccc
tgtgaaatctgtggcacccgtttccggcaccttcagactctgaa
gagccacctgcgaatccacacaggagagaaaccttaccattgtg
agaagtgtaacctgcatttccgtcacaaaagccagctgcgactt
cacttgcgccagaagcatggcgccatcaccaacaccaaggtgca
ataccgcgtgtcagccactgacctgcctccggagctccccaaag
cctgc
The aa sequence of mutant BCL6 (the leucine
mutation is underlined):
(SEQ ID NO: 33)
MASPADSCIQFTRHASDVLLNLNRLRSRDILTDVVIVVSREQFR
AHKTVLMACSGLFYSIFTDQLKCNLSVINLDPEINPEGFCILLD
FMYTSRLNLREGNIMAVMATAMYLQMEHVVDTCRKFIKASEAEM
VSAIKPPREEFLNSRMLMPQDIMAYRGREVVENNLPLRSAPGCE
SRAFAPSLYSGLSTPPASYSMYSHLPVSSLLFSDEEFRDVRMPV
ANPFPKERALPCDSARPVPGEYSRPTLEVSPNVCHSNIYSPKET
IPEEARSDMHYSVAEGLKPAAPSARNAPYFPCDKASKEEERPSS
EDEIALHFEPPNAPLNRKGLVSPQSPQKSDCQPNSPTESCSSKN
ACILQASGSPPAKSPTDPKACNWKKYKFIVLNSLNQNAKPEGLE
QAELGRLSPRAYTAPPACQPPMEPENLDLQSPTKLSASGEDSTI
PQASRLNNIVNRSMTGSPRSSSESHSPLYMHPPKCTSCGSQSPQ
HAEMCLHTAGPTFPEEMGETQSEYSDSSCENGAFFCNECDCRFS
EEASLKRHTLQTHSDKPYKCDRCQASFRYKGNLASHKTVHTGEK
PYRCNICGAQFNRPANLKTHTRIHSGEKPYKCETCGARFVQVAH
LRAHVLIHTGEKPYPCEICGTRFRHLQTLKSHLRIHTGEKPYHC
EKCNLHFRHKSQLRLHLRQKHGAITNTKVQYRVSATDLPPELPK
AC
The nucleotide sequence of mutant BCL6 (the
codon for leucine is underlined):
(SEQ ID NO: 34)
ATGgcctcgccggctgacagctgtatccagttcacccgccatgc
cagtgatgttcttctcaaccttaatcgtctccggagtcgagaca
tcttgactgatgttgtcattgttgtgagccgtgagcagtttaga
gcccataaaacggtcctcatggcctgcagtggcctgttctatag
catctttacagaccagttgaaatgcaaccttagtgtgatcaatc
tagatcctgagatcaaccctgagggattctgcatcctcctggac
ttcatgtacacatctcggctcaatttgcgggagggcaacatcat
ggctgtgatggccacggctatgtacctgcagatggagcatgttg
tggacacttgccggaagtttattaaggccagtgaagcagagatg
gtttctgccatcaagcctcctcgtgaagagttcctcaacagccg
gatgctgatgccccaagacatcatggcctatcggggtcgtgagg
tggtggagaacaacctgccactgaggagcgcccctgggtgtgag
agcagagcctttgcccccagcctgtacagtggcctgtccacacc
gccagcctcttattccatgtacagccacctccctgtcagcagcc
tcctcttctccgatgaggagtttcgggatgtccggatgcctgtg
gccaaccccttccccaaggagcgggcactcccatgtgatagtgc
caggccagtccctggtgagtacagccggccgactttggaggtgt
cccccaatgtgtgccacagcaatatctattcacccaaggaaaca
atcccagaagaggcacgaagtgatatgcactacagtgtggctga
gggcctcaaacctgctgccccctcagcccgaaatgccccctact
tcccttgtgacaaggccagcaaagaagaagagagaccctcctcg
gaagatgagattgccctgcatttcgagccccccaatgcacccct
gaaccggaagggtctggttagtccacagagcccccagaaatctg
actgccagcccaactcgcccacagagtcctgcagcagtaagaat
gcctgcatcctccaggcttctggctcccctccagccaagagccc
cactgaccccaaagcctgcaactggaagaaatacaagttcatcg
tgctcaacagcctcaaccagaatgccaaaccagaggggcTtgag
caggctgagctgggccgcctttccccacgagcctacacggcccc
acctgcctgccagccacccatggagcctgagaaccttgacctcc
agtccccaaccaagctgagtgccagcggggaggactccaccatc
ccacaagccagccggctcaataacatcgttaacaggtccatgac
gggctctccccgcagcagcagcgagagccactcaccactctaca
tgcaccccccgaagtgcacgtcctgcggctctcagtccccacag
catgcagagatgtgcctccacaccgctggccccacgttccctga
ggagatgggagagacccagtctgagtactcagattctagctgtg
agaacggggccttcttctgcaatgagtgtgactgccgcttctct
gaggaggcctcactcaagaggcacacgctgcagacccacagtga
caaaccctacaagtgtgaccgctgccaggcctccttccgctaca
agggcaacctcgccagccacaagaccgtccataccggtgagaaa
ccctatcgttgcaacatctgtggggcccagttcaaccggccagc
caacctgaaaacccacactcgaattcactctggagagaagccct
acaaatgcgaaacctgcggagccagatttgtacaggtggcccac
ctccgtgcccatgtgcttatccacactggtgagaagccctatcc
ctgtgaaatctgtggcacccgtttccggcaccttcagactctga
agagccacctgcgaatccacacaggagagaaaccttaccattgt
gagaagtgtaacctgcatttccgtcacaaaagccagctgcgact
tcacttgcgccagaagcatggcgccatcaccaacaccaaggtgc
aataccgcgtgtcagccactgacctgcctccggagctccccaaa
gcctgc

The immune cells may be any kind of immune cells, including T cells (e.g., regulatory T cells, CD4⁺ T cells, CD8⁺ T cells, alpha beta T cells, gamma-delta T cells, or a mixture thereof), NK cells, invariant NKT cells, NKT cells, innate lymphoid cells, or a mixture thereof. The immune cells may be virus-specific, express a CAR, and/or express a TCR. In some embodiments, the cells are monocytes or granulocytes, e.g., myeloid cells, macrophages, neutrophils, dendritic cells (DCs), mast cells, eosinophils, and/or basophils. Also provided herein are methods of producing and engineering the immune cells as well as methods of using and administering the cells for adoptive cell therapy, in which case the cells may be autologous or allogeneic. Thus, the immune cells may be used as immunotherapy, such as to target cancer cells. These immune cells may be used for therapy as a single cell type or as a combination of multiple immune cell types. In specific embodiments, the immune cells are CD3+, CD4+, CD8+, CD16+, or a mixture thereof.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, such as a partially or fully histocompatibility matched donor or fully histocompatibility mismatched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor may be allogeneic, provided the cells obtained are subject-compatible in that they can be introduced into the subject. Allogeneic donor cells are may or may not be human-leukocyte-antigen (HLA)-compatible.

Additional methods and compositions related to infinite immune cells are described in PCT Patent Application Publication No. WO/2021/034982, incorporated herein by reference in its entirety.

### A. T Cells

In some embodiments, the immune cells are T cells. Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs or PBMCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

In some embodiments, the T cells are derived from the blood, bone marrow, lymph, umbilical cord, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and gamma/delta T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells or stem cell memory cells is carried out to increase efficacy, such as to improve long-term survival, expansion, and/or engraftment following administration, which in some aspects is particularly robust in such sub-populations.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2) or other growth factors.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide or peptides binding to other MHC class I or class II molecules, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells or antigen-presenting cells expressing other HLA molecules. The in vitro-induced T-cells may also be expanded in the absence of antigen-presenting cells.

The autologous T cells can be modified to express a T cell growth or differentiation factor that promotes the growth, differentiation, and activation of the autologous T cells. Suitable T cell growth factors include, for example, interleukin (IL)-2, IL-7, IL-15, IL-18, IL-21, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. In particular aspects, modified autologous T cells express the T cell growth factor at high levels. T cell growth factor coding sequences, such as that of IL-12, are readily available in the art, as are promoters, the operable linkage of which to a T cell growth factor coding sequence promote high-level expression.

### B. NK Cells

In some embodiments, the immune cells are natural killer (NK) cells. NK cells are a subpopulation of lymphocytes that have spontaneous cytotoxicity against a variety of tumor cells, virus-infected cells, and some normal cells in the bone marrow and thymus. NK cells differentiate and mature in the bone marrow, lymph nodes, spleen, tonsils, and thymus. NK cells can be detected by specific surface markers, such as CD16, CD56, and/or CD8 in humans. NK cells do not express T cell antigen receptors, the pan T marker CD3, or surface immunoglobulin B cell receptors.

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, tissues, or umbilical cord blood by methods well known in the art.

### C. NKT Cells

Natural killer T (NKT) cells are a heterogeneous group of T cells that share properties of both T cells and natural killer cells. Many of these cells recognize the non-polymorphic CD1d molecule, an antigen-presenting molecule that binds self and foreign lipids and glycolipids. They constitute only approximately 0.1% of all peripheral blood T cells. NKT cells are a subset of T cells that coexpress an αβ T-cell receptor, but also express a variety of molecular markers that are typically associated with NK cells, such as NK1.1. Invariant natural killer T (iNKT) cells express high levels of and are dependent on the transcriptional regulator promyelocytic leukemia zinc finger for their development. Currently, there are five major distinct iNKT cell subsets. These subset cells produce a different set of cytokines once activated. The subtypes iNKT1, iNKT2 and iNKT17 mirror Th cell subsets in cytokine production. In addition, there are subtypes specialized in T follicular helper-like function and IL-10 dependent regulatory functions.

### D. Innate Lymphoid Cells

Innate lymphoid cells (ILCs) are a group of innate immune cells that are derived from common lymphoid progenitor (CLP) and belong to the lymphoid lineage. These cells are defined by absence of antigen specific B or T cell receptor because of the lack of recombination activating gene (RAG). ILCs do not express myeloid or dendritic cell markers. They play a role in protective immunity and the regulation of homeostasis and inflammation, so their dysregulation can lead to immune pathology such as allergy, bronchial asthma and autoimmune disease. ILCs can be divided based on the cytokines that they can produce, and the transcription factors that regulate their development and function.

### V. Formulations and Culture of Cells

In particular embodiments, cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21TM supplement, or combinations thereof. In some embodiments, the medium comprises or futher comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, ug/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

### A. Cells

Certain embodiments relate to cells comprising polypeptides or nucleic acids of the disclosure. In some embodiments the cell is an immune cell or a T cell. “T cell” includes all types of immune cells expressing CD3 including T-helper cells, invariant natural killer T (INKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### VI. General Pharmaceutical Compositions

In some embodiments, pharmaceutical compositions are administered to a subject. Different aspects may involve administering an effective amount of a composition to a subject. In some embodiments, a cellular therapy (e.g., dual CAR T cells) is administered to the subject to protect against or treat a condition (e.g., cancer). Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### REFERENCES

The following references, to the extent that they provide exemplary procedural or other details supplementary to those set forth herein, are specifically incorporated herein by reference.
1. Kochenderfer J N, Dudley M E, Kassim S H, et al. Chemotherapy-refractory diffuse large B-cell lymphoma and indolent B-cell malignancies can be effectively treated with autologous T cells expressing an anti-CD19 chimeric antigen receptor. J Clin Oncol. 2015; 33(6):540-549.
2. Sotillo E, Barrett D M, Black K L, et al. Convergence of Acquired Mutations and Alternative Splicing of CD19 Enables Resistance to CART-19 Immunotherapy. Cancer Discov. 2015; 5(12):1282-1295.
3. Neelapu S S, Locke F L, Bartlett N L, et al. Axicabtagene Ciloleucel CAR T-Cell Therapy in Refractory Large B-Cell Lymphoma. N Engl J Med. 2017; 377(26):2531-2544.
4. Plaks V, Rossi J M, Chou J, et al. CD19 target evasion as a mechanism of relapse in large B-cell lymphoma treated with axicabtagene ciloleucel. Blood. 2021.
5. Neelapu S S, Rossi J M, Jacobson C A, et al. CD19-Loss with Preservation of Other B Cell Lineage Features in Patients with Large B Cell Lymphoma Who Relapsed Post-Axi-Cel. Blood. 2019; 134 (Supplement_1):203-203.
6. Ormhøj M, Scarfo I, Cabral M L, et al. Chimeric Antigen Receptor T Cells Targeting CD79b Show Efficacy in Lymphoma with or without Cotargeting CD19. Clin Cancer Res. 2019; 25 (23): 7046-7057.
7. Baird J H, Frank M J, Craig J, et al. CD22-directed CAR T-cell therapy induces complete remissions in CD19-directed CAR-refractory large B-cell lymphoma. Blood. 2021; 137(17):2321-2325.
8. Jiang D, Tian X, Bian X, Zhu T, Qin H, Zhang R, Xu Y, Pan Z, Huang H, Fu J, Wu D, Chu J. T cells redirected against Igβ for the immunotherapy of B cell lymphoma. Leukemia. 2020 March; 34(3):821-830. PMID: 31624374.
9. Ding S, Mao X, Cao Y, Wang N, Xu H, Zhou J. Targeting CD79b for Chimeric Antigen Receptor T-Cell Therapy of B-Cell Lymphomas. Target Oncol. 2020 June; 15(3):365-375. PMID: 32495161.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Design and Use of Dual CD19−CD79b CAR T Cells

### Methods

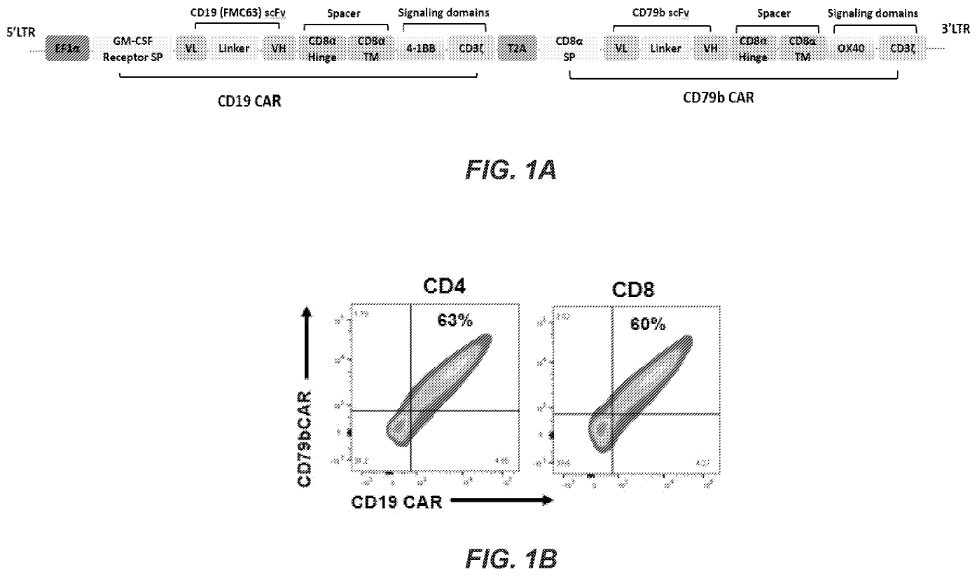

Generation of dual CD19−CD79b CAR constructs. The dual CD19−CD79b CAR described in this embodiment is composed of two second-generation CARs, an anti-CD19 CAR (murine anti-CD19 FMC63) and an anti-CD79b CAR (murine anti-CD79b 28B clone) linked by the T2A self-cleaving peptide. CD19 CAR contains CD8α hinge and transmembrane domain with 4-1BB costimulatory domain and CD3ζ ntracellular domains. CD79b CAR comprises CD8α hinge and transmembrane domain with OX40 costimulatory domain and CD35ζ activation domains. The two CAR constructs were cloned into a bicistronic third-generation lentiviral expression vector under the control of human EF1α promoter. See FIG. 1A.

Transduction of lentivirus containing dual CD19−CD79b CAR in primary T cells. Primary T cells were enriched from peripheral blood mononuclear cells (PBMC) obtained from normal donors. Purified T cells were activated by antibodies against CD3, CD28, and CD2 and cultured for 60 to 72 hours with IL-2. The activated T cells were transduced by an RD114-pesudeotyped lentivirus containing dual CD19−CD79b CAR. Expression of CD19 CAR and CD79b CAR were assessed by flow cytometry by staining with CD19 protein-conjugated FITC and CD79b protein-conjugated PE.

Degranulation assay. Untransduced T cells, CD19 CAR T cells, and dual CD19−CD79b CAR T cells were co-cultured with tumor cells from Daudi, SUDHL6, PDX203, PDX300, NALM6, Daudi-CD19KO, SUDHL6−CD19KO, and K562 cell lines, respectively for 5 to 6 hours. Monensin (0.7 μL/mL) and anti-CD107a antibody conjugated to APC were added to culture medium at start of co-culture. Degranulation was assessed by flow cytometry by determining CD107a expression in T cells at the end of culture period.

Cytotoxicity and proliferation assays. Untransduced T, CD19 CAR T, and dual CD19−CD79b CAR T were labeled with Cell Trace™ Far Red and tumor cell lines (Daudi, SUDHL6, PDX203, PDX300, Daudi-CD19KO, SUDHL6−CD19KO, NALM6 and K562) were labeled with Cell Trace™ Violet. Effector cells were co-cultured with tumor cells at effector:target ratios of 1:2, 1:1, and 2:1. Untransduced T and tumor cells alone were used as controls. The percentage of live and dead cells and T cell proliferation were analyzed by flow cytometry at four time points (day0, day1, day2 and day4) after staining with Aqua-live/dead, PerCP-Cy5.5-CD4, PE-CF594-CD8, CD19 protein-conjugated FITC, and CD79b protein-conjugated PE. Absolute number of live tumor cells was calculated using CountBright™ absolute counting beads (ThermoFisher Scientific).

### Results

Generation of dual CD19−CD79b CAR T cells. A bicistronic CD19−CD79b CAR construct was created that can be used to simultaneously express two CARs on the surface of primary T cells to target CD19 and CD79b antigens (FIG. 1A). By staining with CD19 protein-conjugated FITC and CD79b protein-conjugated PE about 3 days after lentiviral transduction, high expression of both CD19 and CD79b CARs was shown on the surface of T cells indicating high transduction efficiency (FIG. 1B).

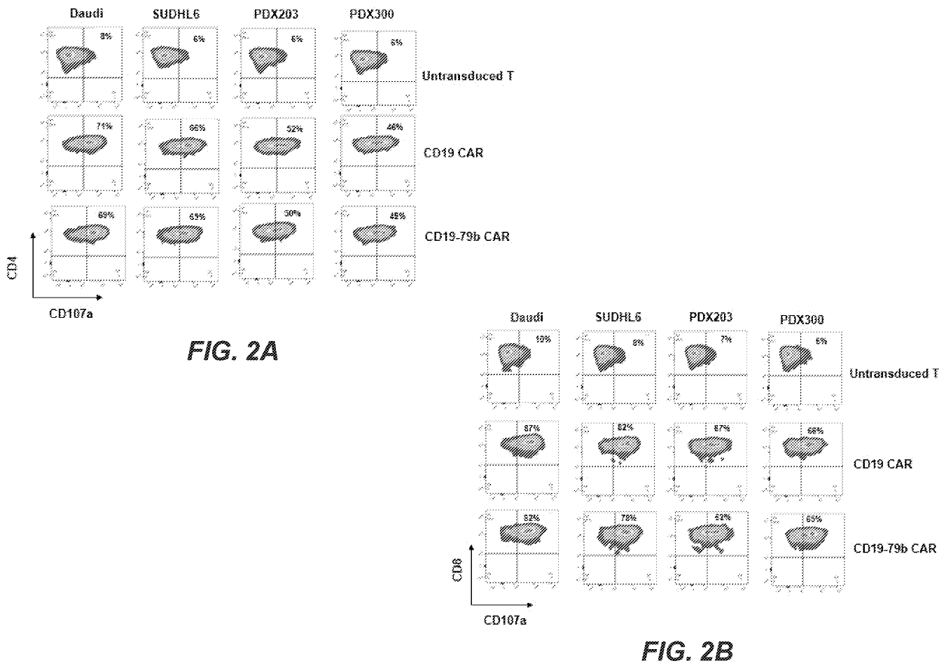

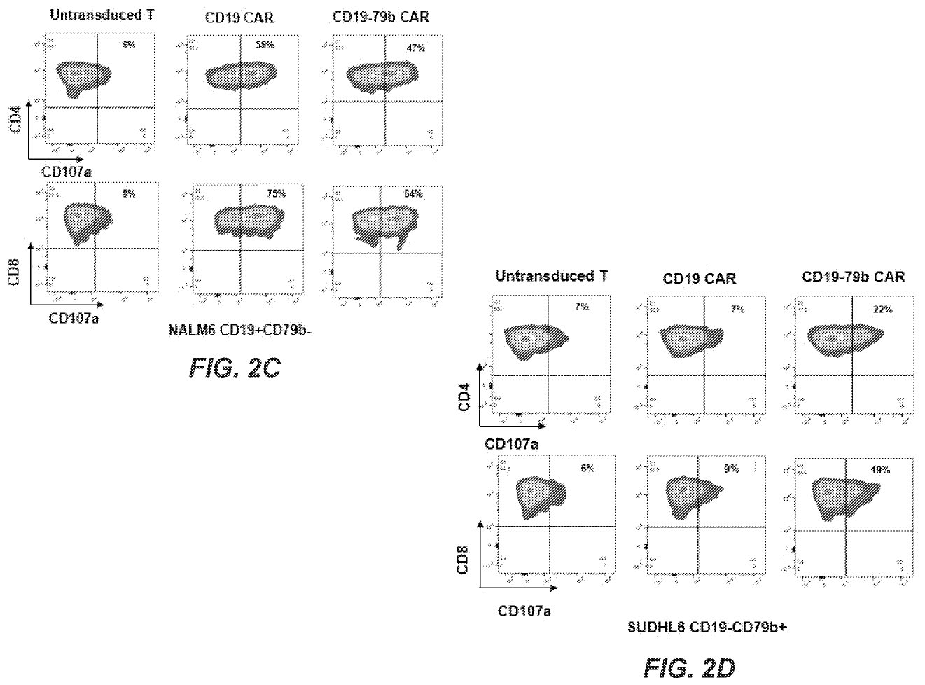

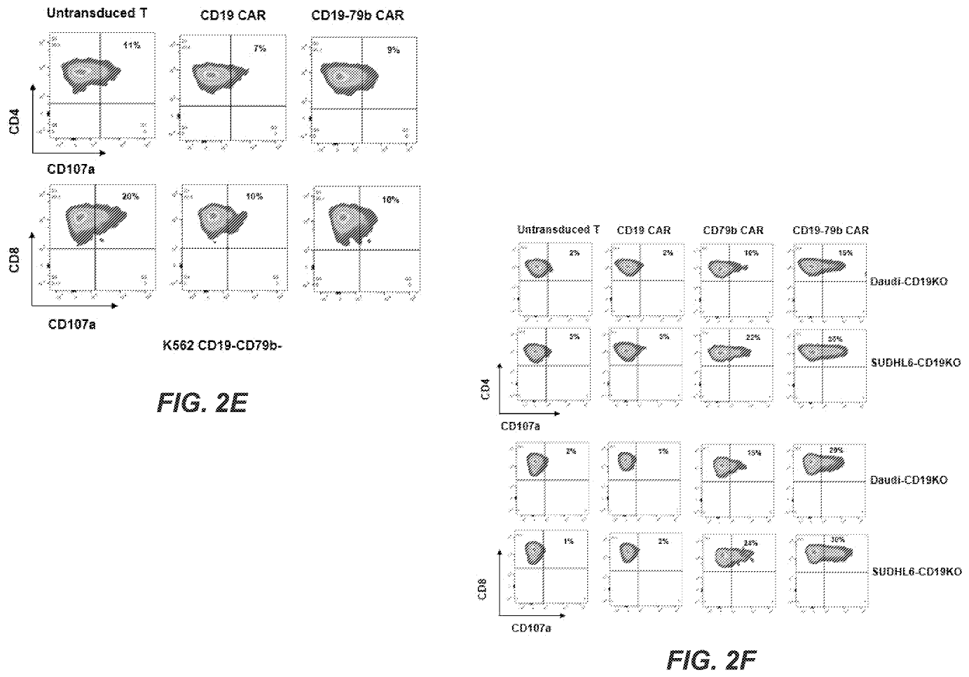

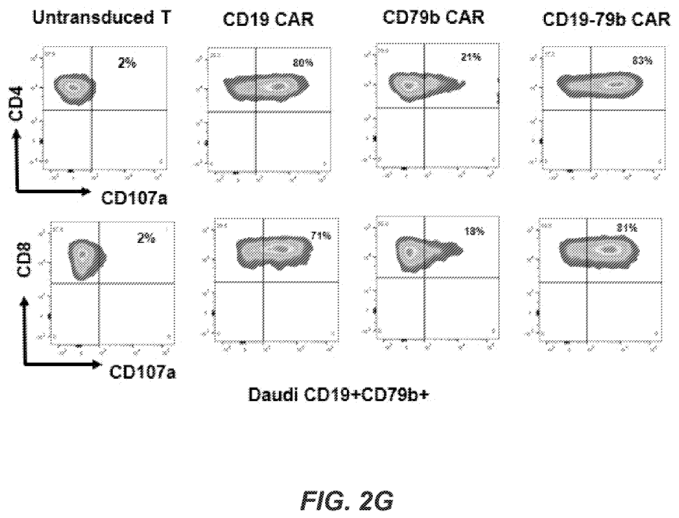

Dual CD19−CD79b CAR T cells degranulate in response to B-cell lymphoma and leukemia cell lines in CD19 or CD79b dependent manner. Degranulation is an indirect measure of perforin-granzyme-mediated killing of target cells by T cells. High CD107a expression was observed in dual CD19−CD79b CAR T cells when they were co-cultured with B-cell lymphoma/leukemia tumor cell lines as fresh (FIGS. 2A-2E) or cryopreserved and thawed T cells (FIGS. 2F-2G). Importantly, the dual CAR T cells recognized CD19+CD79b+, CD19−CD79b+, and CD19+CD79b− tumor cells but not CD19−CD79b− tumor cells suggesting that both the CARs expressed on cell surface are functional and the degranulation required expression of one or the other target antigen.

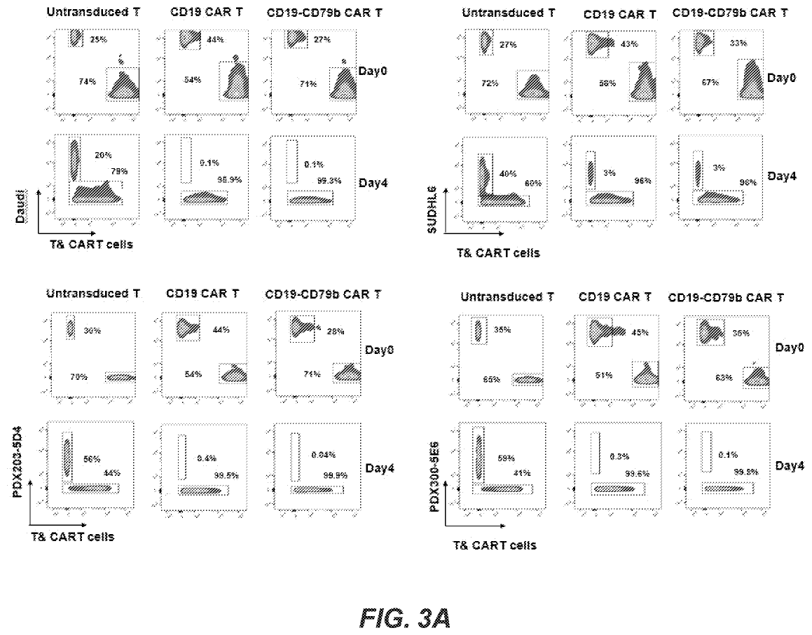

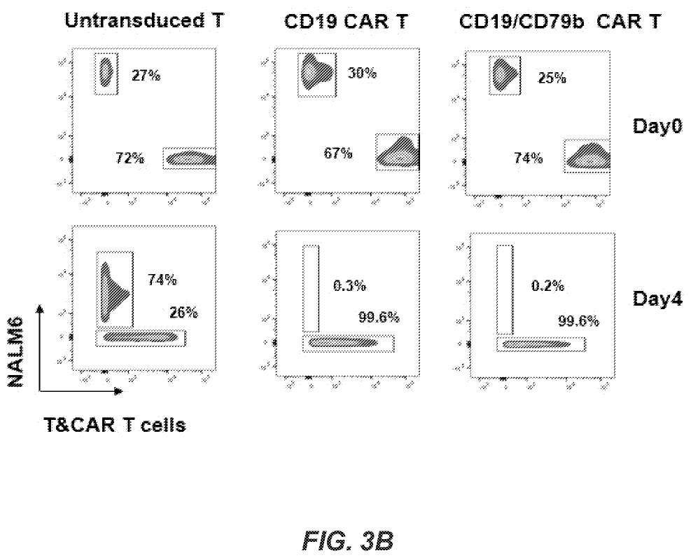

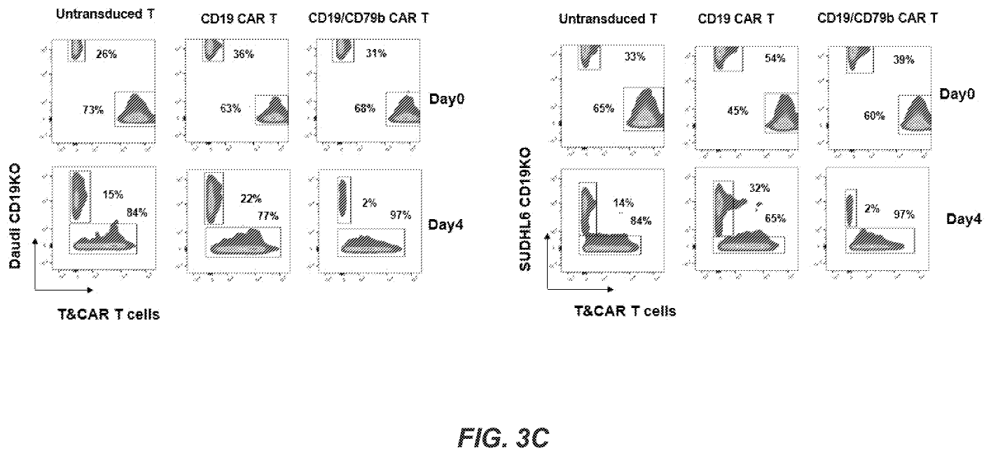

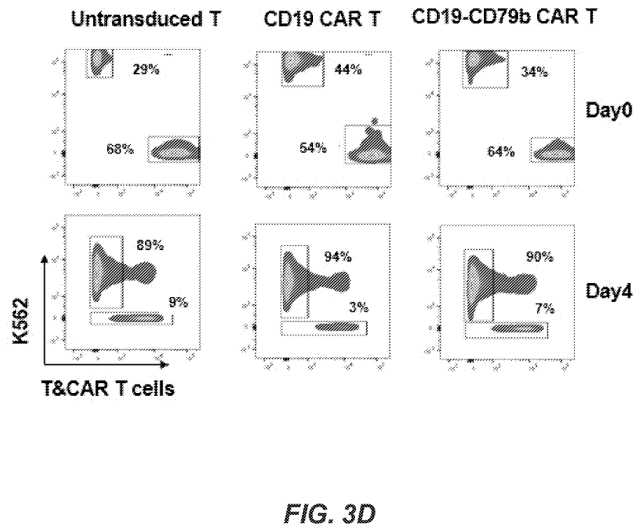

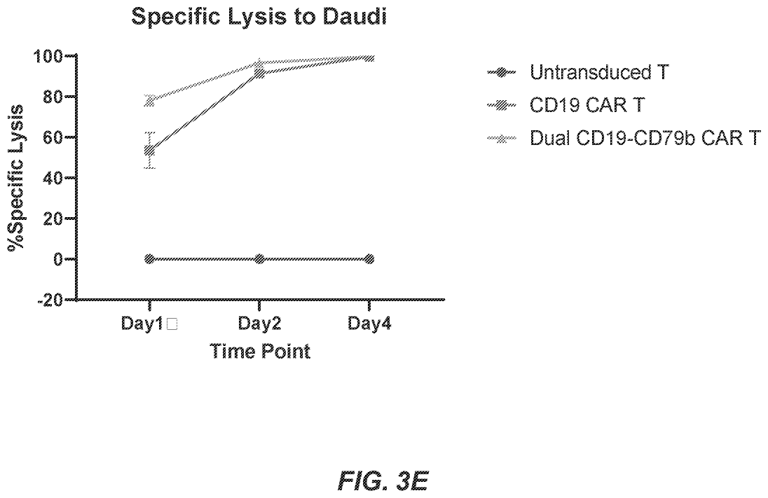

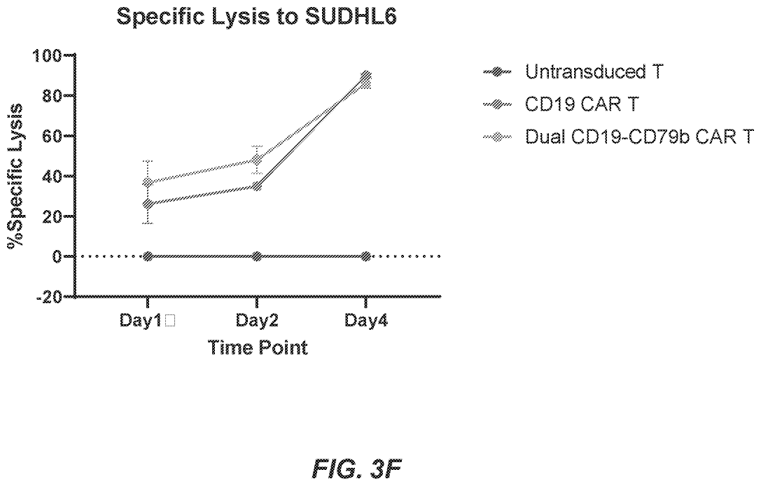

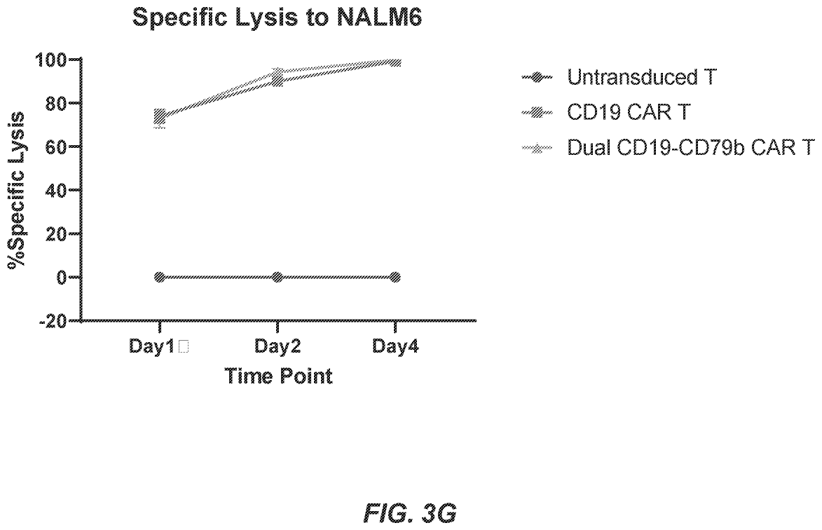

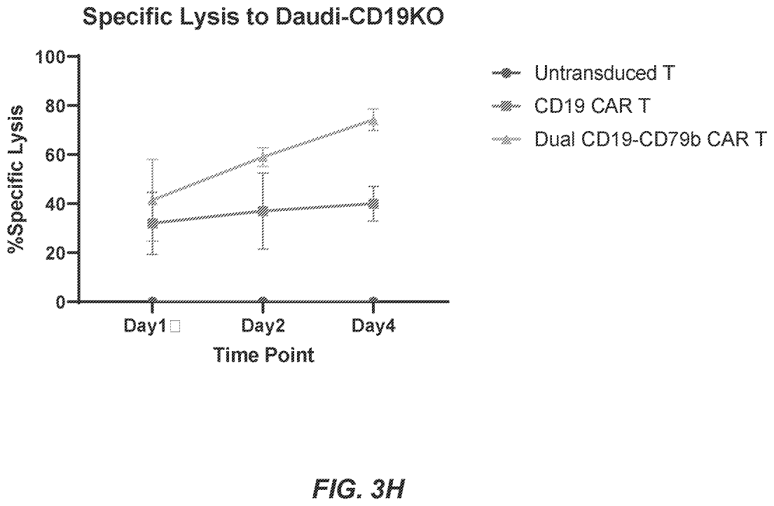

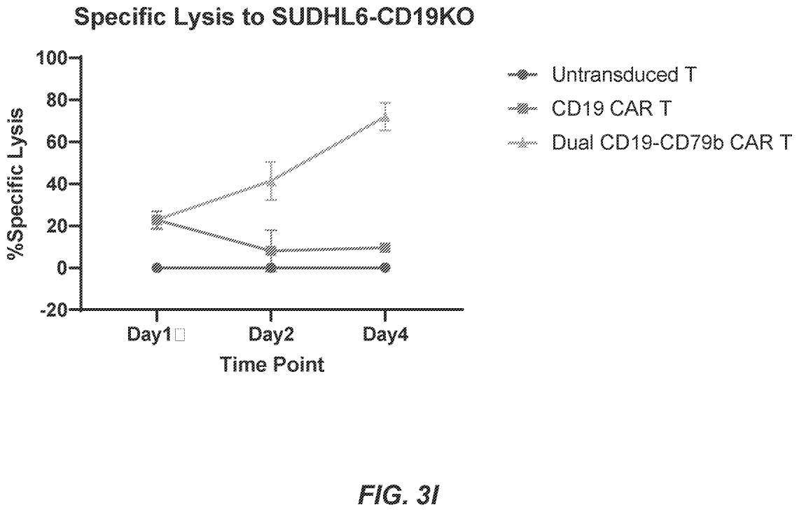

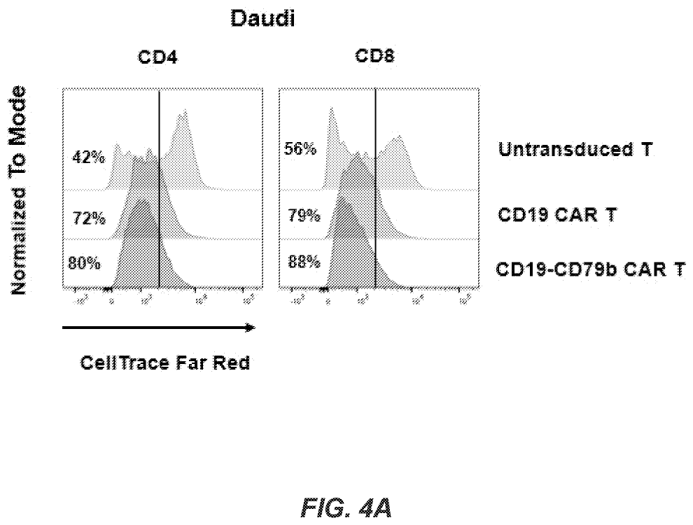

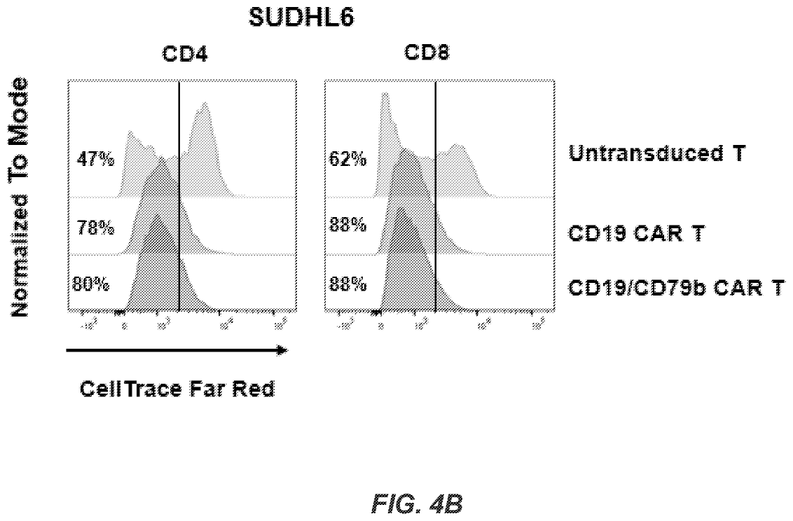

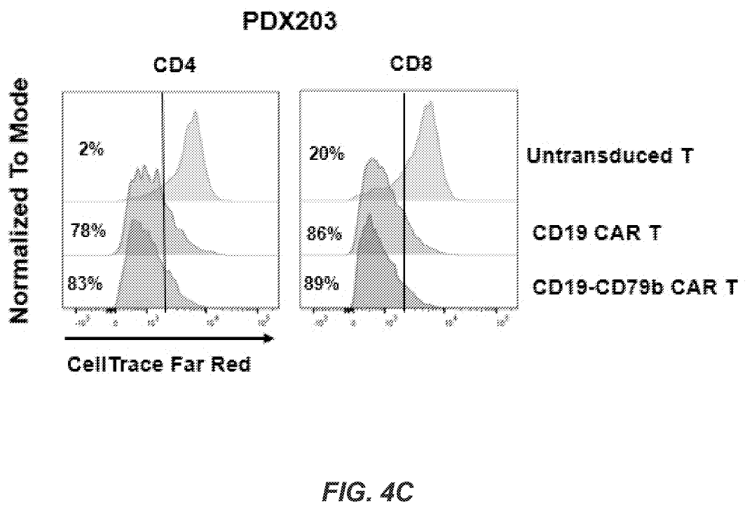

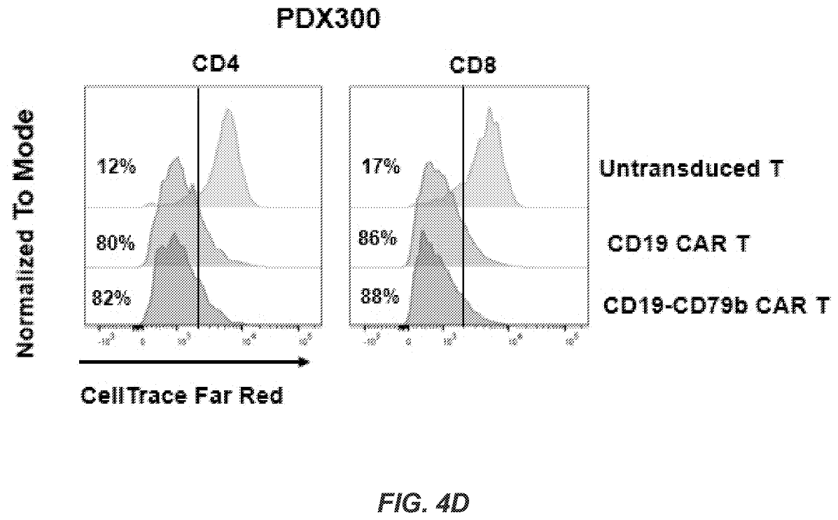

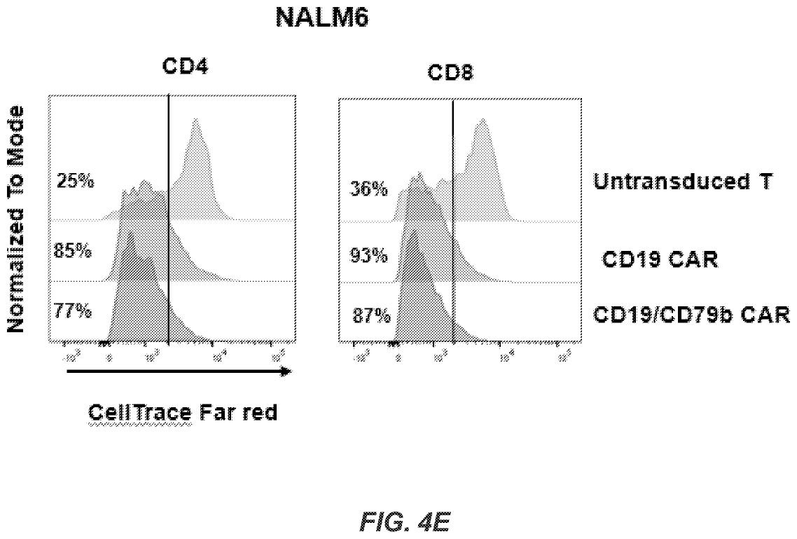

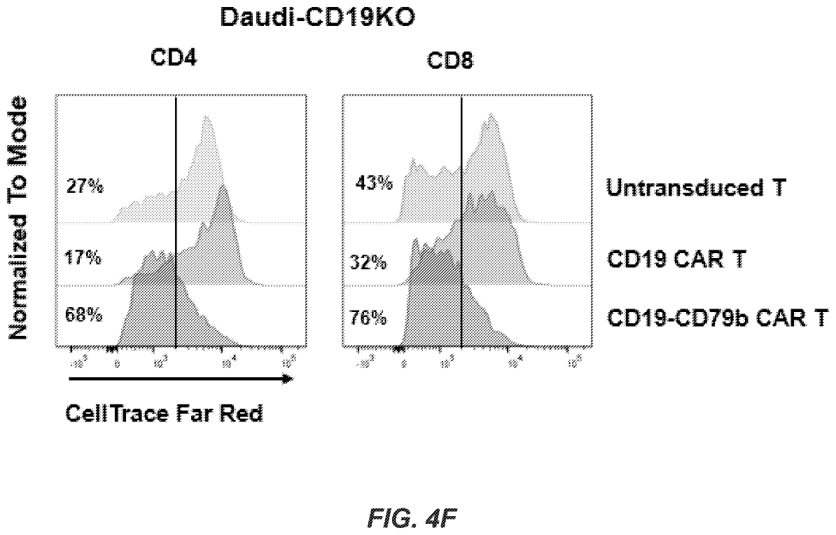

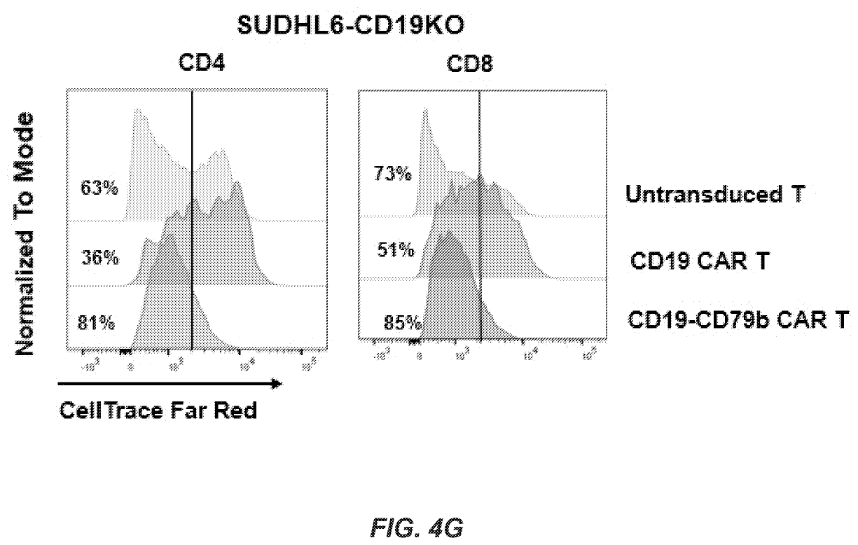

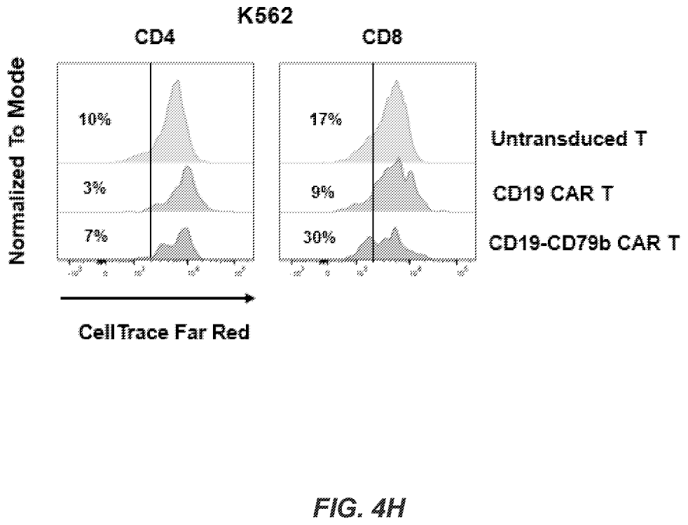

Dual CD19−CD79b CAR T cells are cytotoxic to B-cell leukemia and lymphoma cell lines in CD19 or CD79b dependent manner. Similar to the degranulation assay, dual CD19−CD79b CAR T cells specifically recognized and induced lysis of CD19+CD79b+ (Daudi, SUDHL6, PDX203, and PDX300) (FIG. 3A), CD19+CD79b− (NALM6) (FIG. 3B), and CD19−CD79b+ (Daudi-CD19KO and SUDHL6−CD19KO) tumor cells (FIG. 3C) but not CD19−CD79b− tumor cells (K562) (FIG. 3D). Percent specific lysis by CAR T cells for each tumor cell line was calculated as follows and normalized to untransduced T cells: % Specific Lysis=1−[live target cells with CAR T cells/live target cells with untransduced T cells] (FIGS. 3E-3I).

Dual CD19−CD79b CAR T cells proliferate in response to B-cell leukemia and lymphoma cell lines in a CD19 or CD79b dependent manner. After co-culture for 4 days, it was observed that dual CD19−CD79b CAR T cells proliferated when co-cultured with B-cell leukemia and lymphoma tumor cell lines (Daudi, SUDHL6, PDX203, PDX300, Daudi-CD19KO, SUDHL6−CD19KO, and NALM6).

### Example 2—Selection of Dual CAR Construct

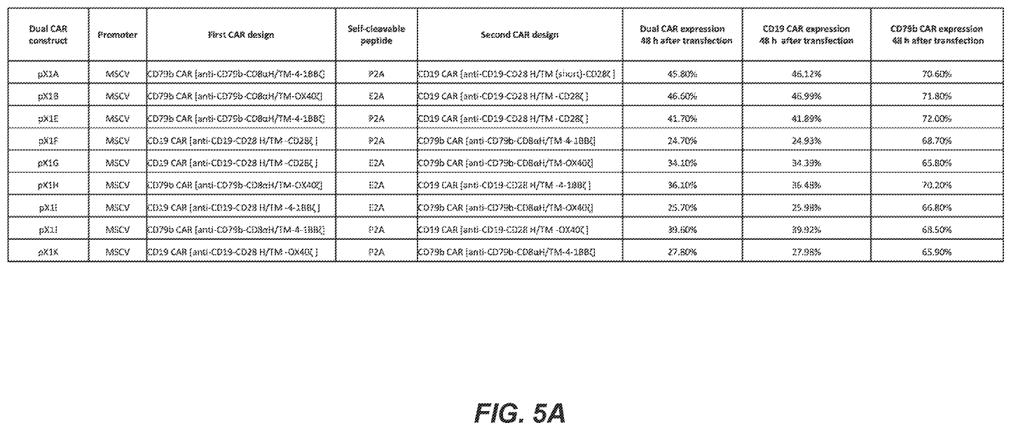

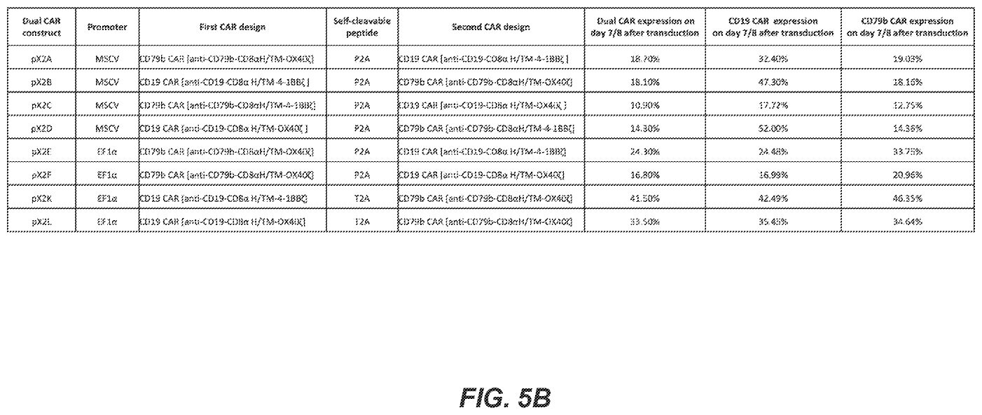

Several different designs of dual CD19−CD79b CAR construct were generated with various permutations of promoters (MSCV or EF1a), self-cleaving peptides (E2A, P2A, or T2A), order of CD19 vs. CD79b CAR, hinge/transmembrane (H/TM) domains (CD8a or CD28), and costimulatory domains (CD28, 4-1BB, or OX-40). All constructs had CD3 zeta (ζ) as signaling domain. The dual CAR constructs were then either transfected into 293T cells or lentivirally transduced into primary T cells. Transfection or transduction efficiency was assessed by flow cytometry after 48 hours in 293T cells and after 7 or 8 days in primary T cells, respectively. Percentages of cells positive for CD19 or CD79b CAR or both are shown in FIGS. 5A and 5B. The data show that most dual CAR constructs resulted in unequal expression of one or the other CAR with the exception of pX2K and pX2L, which gave comparable expression for each of the CARs with pX2K resulting in higher transduction efficiency than pX2L. Based on these results pX2K dual CAR construct was chosen for further development. Results are representative of at least two experiments for the various constructs.

### Example 3—CD19−CD79b dual CAR T Cells Produced Effector Cytokines in Response to B-cell Lymphoma and Leukemia Cell Lines in CD19 or CD79b Dependent Manner

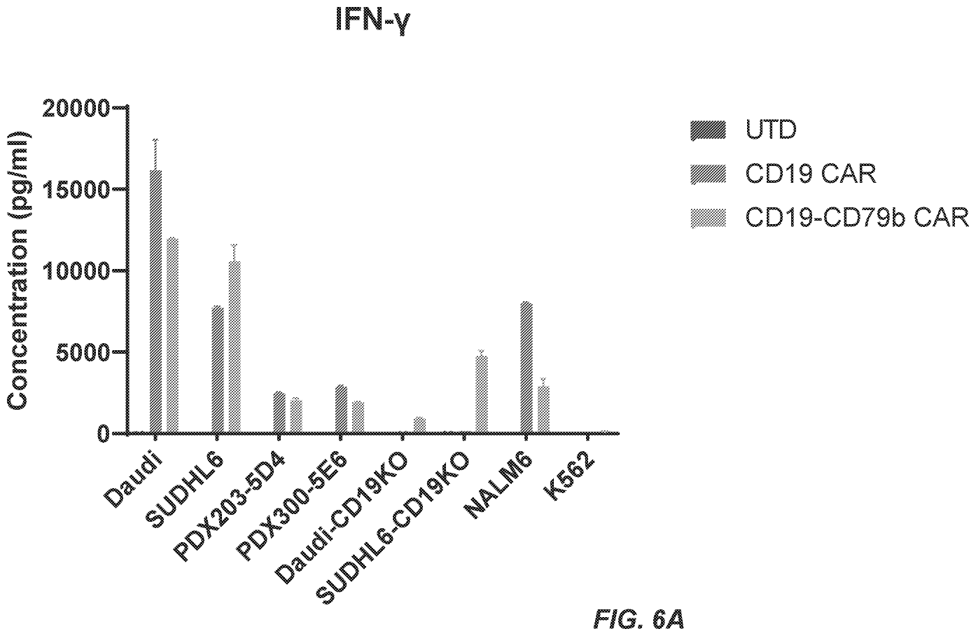

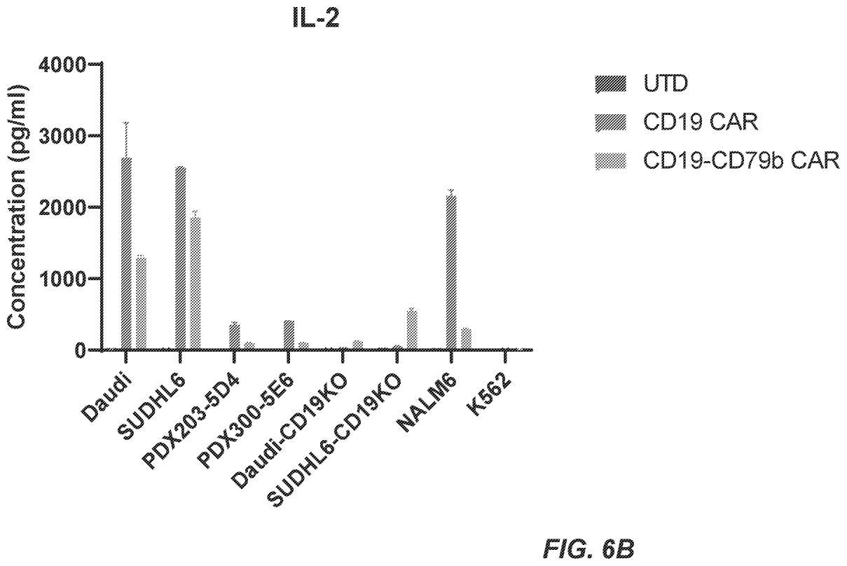

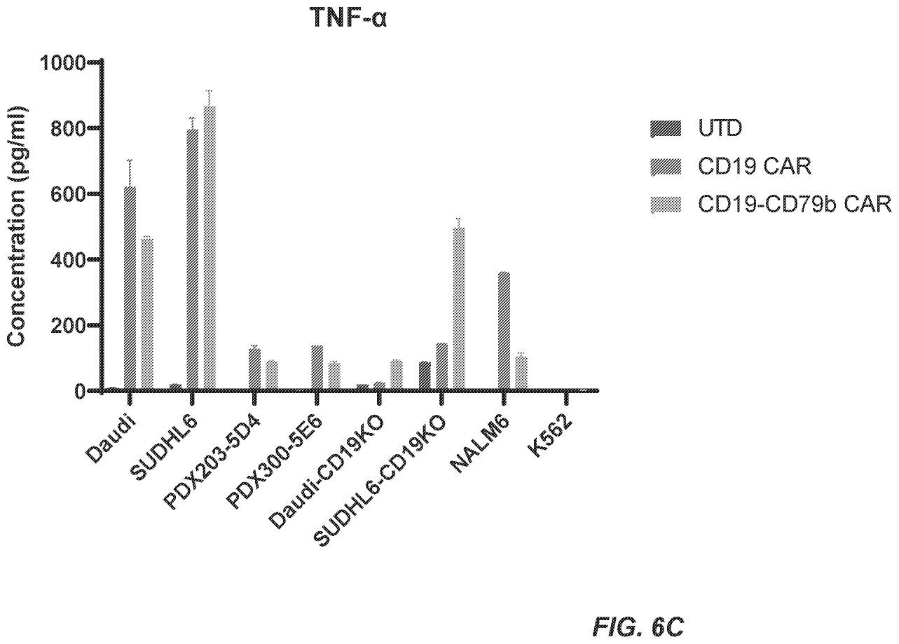

Dual CD19−CD79b CAR T cells specifically recognized and released IFN-γ (FIG. 6A (here, UTD is zero for all lines), IL-2 (FIG. 6B), and TNF-α (FIG. 6C) in response to B-cell lymphoma cell lines Daudi, SUDHL6, PDX203-5D4, and PDX300-5E6 that are CD19+CD79b+, B-cell leukemia cell line NALM6 that is CD19+CD79b−, and Daudi-CD19KO and SUDHL6−CD19KO lymphoma cell lines that are CD19−CD79b+, but not in response to CD19−CD79b− K562 tumor cells. CD19 CAR T cells were used as a control.

### Example 4—CD19−CD79b Dual CAR T Cells Have Potent Antitumor Activity In Vivo in a NALM6 CD19+CD79B− Cell Line Xenograft Model

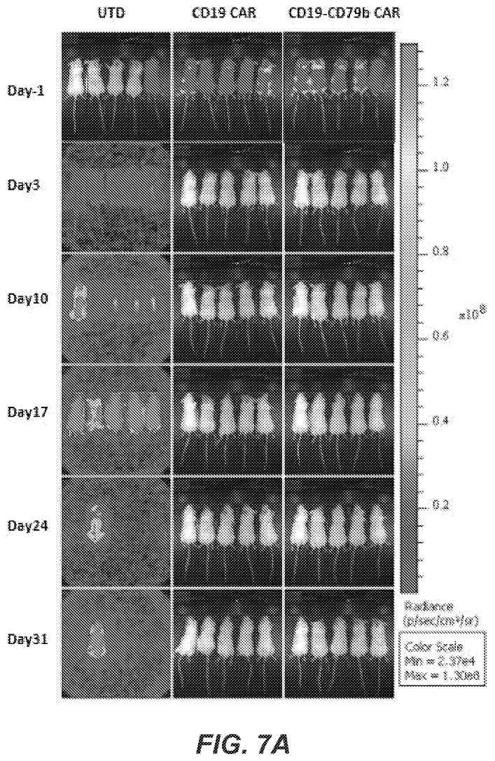

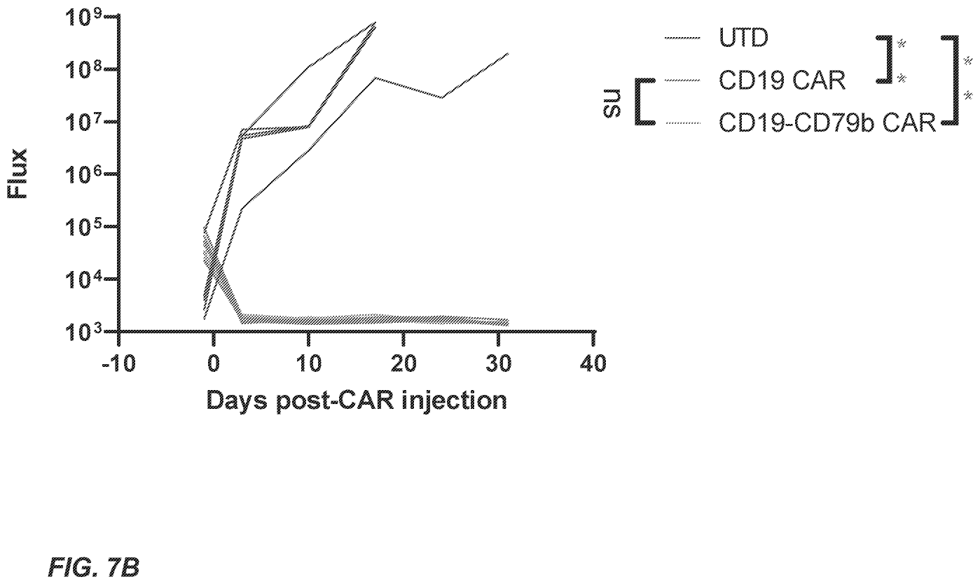

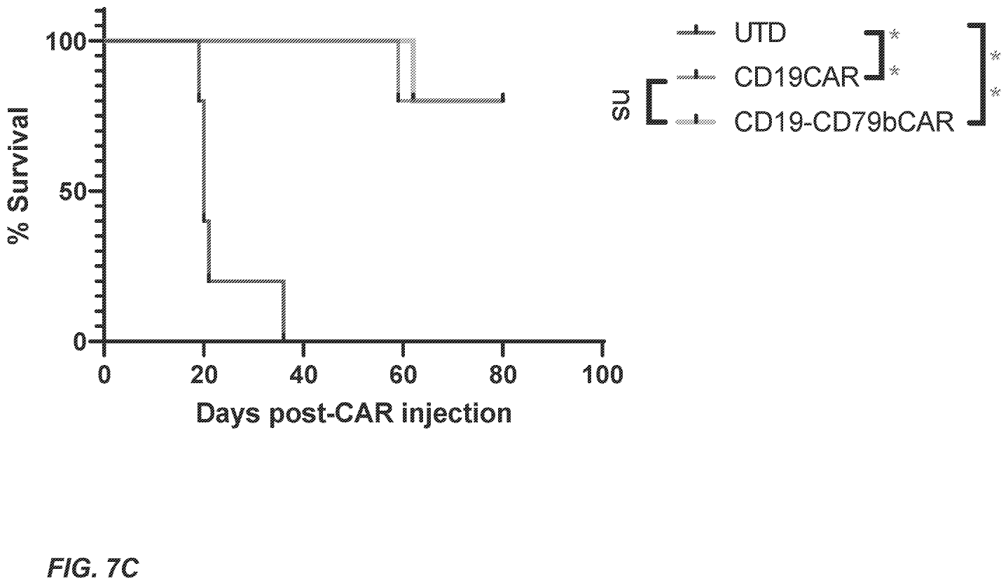

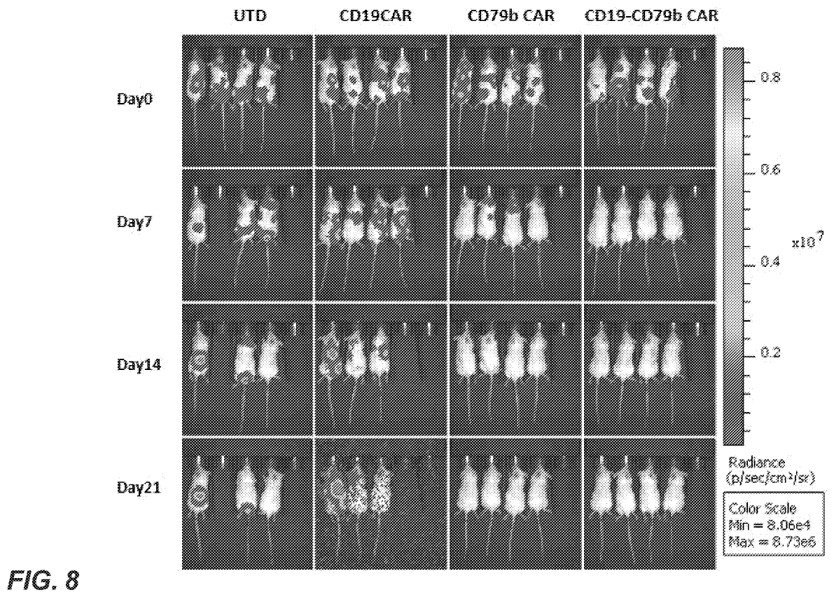

NOD.Cg-Prkdc^{scid}Il2rg^{tm1Wjl}/SzJ (NSG) mice were injected intravenously with 0.2×10⁶ firefly luciferase-expressing NALM6 tumor cells that are CD19+CD79b−. After 4 days, 5×10⁶ CAR+ cells/mouse of CD19CAR T or CD19−CD79b dual CAR T cells, and corresponding untransduced T cells were injected into tumor-bearing mice via tail vein injection. Tumor burden was measured by bioluminescence imaging at the indicated days (FIG. 7A) and quantified by total flux (n=5 mice per group, significance among treatment groups was determined by Mann-Whitney test. p=ns, **, p<0.01) (FIG. 7B). Kaplan-Meier survival curve for mice treated with untransduced T, CD19 CAR T and CD19−CD79b dual CAR T (n=5 mice per group). Significance among treatment groups was determined by log-rank (Mantel-Cox) test (p=ns, **, p<0.01) (FIG. 7C). The antitumor activity was comparable to CD19 CAR T cells indicating that the CD19 targeting CAR within the dual CAR T cells was functional.

### Example 5—CD19−CD79b Dual CAR T Cells Have Potent Antitumor Activity In Vivo in a Patient-Derived Xenograft Model with PDX203-5D4 CD19KO that is CD19−CD79b+.

NOD.Cg-Prkdc^{scid}Il2rg^{tm1Wjl}/SzJ (NSG) mice were injected intravenously with 0.2×10⁶ firefly luciferase-expressing PDX203-5D4 CD19KO cells. After 15 days, 3×10⁶ CAR+ cells/mouse of CD19CAR T, CD79b CAR T, or CD19−CD79b dual CAR T cells, and corresponding untransduced T cells were injected via tail vein into tumor-bearing mice. Tumor burden was evaluated by bioluminescence imaging on day 0, day 7, day 14, and day 21.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A polynucleotide comprising:
(a) a first sequence encoding an anti-CD19 chimeric antigen receptor (CAR), the anti-CD19 CAR comprising:
(i) a GM-CSF Receptor alpha signal peptide;
(ii) a CD19-binding domain;
(iii) a CD8α hinge region;
(iv) a CD8α transmembrane domain;
(v) a 4-1BB signaling domain; and
(vi) a CD3 zeta signaling domain; and
(b) a second sequence encoding an anti-CD79b CAR, the anti-CD79b CAR comprising:
(i) a CD8α signal peptide;
(ii) a CD79b-binding domain;
(iii) a CD8α hinge region;
(iv) a CD8α transmembrane domain;
(v) a OX40 signaling domain; and
(vi) a CD3 zeta signaling domain.

2. The polynucleotide of claim 1, wherein the first sequence is 5′ relative to the second sequence.

3. The polynucleotide of claim 1 or 2, further comprising a third sequence encoding a T2A peptide.

4. The polynucleotide of claim 3, wherein the third sequence is 3′ relative to the first sequence and 5′ relative to the second sequence.

5. The polynucleotide of claim 3, wherein the T2A peptide comprises SEQ ID NO:9.

6. The polynucleotide of any of claims 1-5, wherein the GM-CSF Receptor signal peptide comprises SEQ ID NO:1.

7. The polynucleotide of any of claims 1-6, wherein the CD19-binding domain is an scFv of an anti-CD19 antibody.

8. The polynucleotide of claim 7, wherein the CD19-binding domain comprises a VL having SEQ ID NO:2 and a VH having SEQ ID NO:4.

9. The polynucleotide of claim 8, wherein the CD19-binding domain further comprises a linker linking the VL and the VH.

10. The polynucleotide of claim 9, wherein the linker comprises SEQ ID NO:3.

11. The polynucleotide of any of claims 1-10, wherein the CD8α hinge region of (a) comprises SEQ ID NO:5.

12. The polynucleotide of any of claims 1-11, wherein the CD8α transmembrane domain of (a) comprises SEQ ID NO:6.

13. The polynucleotide of any of claims 1-12, wherein the 4-1BB signaling domain comprises SEQ ID NO:7.

14. The polynucleotide of any of claims 1-13, wherein the CD3 zeta signaling domain of (a) comprises SEQ ID NO:8.

15. The polynucleotide of any of claims 1-14, wherein the CD8α signal peptide comprises SEQ ID NO:10.

16. The polynucleotide of any of claims 1-15, wherein the CD79b-binding domain is an scFv of an anti-CD79 antibody.

17. The polynucleotide of claim 16, wherein the CD79b-binding domain comprises a VL having SEQ ID NO:11 and a VH having SEQ ID NO:13.

18. The polynucleotide of claim 17, wherein the CD79b-binding domain further comprises a linker linking the VL and the VH.

19. The polynucleotide of claim 18, wherein the CD79b-binding domain comprises a linker having SEQ ID NO:12,

20. The polynucleotide of any of claims 1-19, wherein the CD8α hinge region of (b) comprises SEQ ID NO:5.

21. The polynucleotide of any of claims 1-20, wherein the CD8α transmembrane domain of (b) comprises SEQ ID NO:6.

22. The polynucleotide of any of claims 1-21, wherein the OX40 signaling domain comprises SEQ ID NO:14.

23. The polynucleotide of any of claims 1-22, wherein the CD3 zeta signaling domain of (b) comprises SEQ ID NO:8.

24. The polynucleotide of any of claims 1-23, wherein the polynucleotide comprises a sequence encoding a polypeptide having SEQ ID NO:15.

25. The polynucleotide of claim 24, wherein the polynucleotide comprises a sequence having at least 90% identity with SEQ ID NO:30.

26. The polynucleotide of any of claims 1-25, further comprising a promoter.

27. The polynucleotide of claim 26, wherein the promoter is an EF1α promoter.

28. A vector comprising the polynucleotide of any of claims 1-27.

29. A polypeptide encoded by the polynucleotide of any of claims 1-25 or the vector of claim 28.

30. A method of generating an engineered cell comprising introducing into the cell the polynucleotide of claims 1-27, the vector of claim 28, or the polypeptide of claim 29.

31. An engineered cell comprising the polynucleotide of any of claims 1-25 or the vector of claim 28.

32. The engineered cell of claim 31, wherein the engineered cell is a T cell.

33. The engineered cell of claim 31, wherein the engineered cell is a natural killer (NK) cell.

34. The engineered cell of claim 31, wherein the engineered cell is a macrophage.

35. A population of engineered cells comprising the engineered cell of any of claims 31-33.

36. The population of engineered cells of claim 35, wherein at least 20% of the engineered cells in the population express both the anti-CD19 CAR and the anti-CD79b CAR.

37. The population of engineered cells of claim 35 or claim 36, wherein the population of engineered cells comprises a subset that express either the anti-CD19 CAR or the anti-CD79b CAR, wherein at least 80% of the subset express both the anti-CD19 CAR and the anti-CD79b CAR.

38. A pharmaceutical composition comprising the population of engineered cells of any of claims 35-37 and an excipient.

39. A method for treating a subject for cancer, the method comprising administering to the subject a therapeutically effective amount of the pharmaceutical composition of claim 38.

40. The method of claim 39, wherein the subject has a B-cell malignancy.

41. The method of claim 40, wherein the subject has diffuse large B-cell lymphoma, high-grade B-cell lymphoma, follicular lymphoma, marginal zone lymphoma, mantle cell lymphoma, Burkitt lymphoma, lymphoplasmacytic lymphoma, hairy cell leukemia, or chronic lymphocytic leukemia.

42. The method of any of claims 39-41, wherein the subject has CD19⁺CD79b⁺ cancer.

43. The method of any of claims 39-41, wherein the subject has CD19⁻CD79b⁺ cancer.

44. The method of any of claims 39-41, wherein the subject has CD19⁺CD79b⁻ cancer.

45. A polynucleotide comprising, in order from 5′ to 3′:
(a) a first sequence encoding an anti-CD19 chimeric antigen receptor (CAR), the anti-CD19 CAR comprising:
(i) a GM-CSF Receptor signal peptide comprising SEQ ID NO:1;
(ii) a CD19-binding domain comprising SEQ ID NO:2, SEQ ID NO:3, and SEQ ID NO:4;
(iii) a CD8α hinge region comprising SEQ ID NO:5;
(iv) a CD8α transmembrane domain comprising SEQ ID NO:6;
(v) a 4-1BB signaling domain comprising SEQ ID NO:7; and
(vi) a CD3 zeta signaling domain comprises SEQ ID NO:8;
(b) a second sequence encoding a T2A peptide comprising SEQ ID NO:9; and
(c) a third sequence encoding an anti-CD79b CAR, the anti-CD79b CAR comprising:
(i) a CD8α signal peptide comprising SEQ ID NO:1-;
(ii) a CD79b-binding domain comprising SEQ ID NO:11, SEQ ID NO: 12, and SEQ ID NO:13;
(iii) a CD8α hinge region comprising SEQ ID NO:5;
(iv) a CD8α transmembrane domain comprising SEQ ID NO:6;
(v) a OX40 signaling domain comprising SEQ ID NO:14; and
(vi) a CD3 zeta signaling domain comprises SEQ ID NO:8.

46. The polynucleotide of claim 45, wherein the polynucleotide comprises a sequence encoding a polypeptide having SEQ ID NO:15.

47. The polynucleotide of claim 46, wherein the polynucleotide comprises a sequence having at least 90% identity with SEQ ID NO:30.

48. The polynucleotide of claim 47, wherein the polynucleotide comprises SEQ ID NO: 30.

49. The polynucleotide of any of claims 45-48, further comprising a promoter.

50. The polynucleotide of claim 49, wherein the promoter is an EF1α promoter

51. A vector comprising the polynucleotide of any of claims 45-48.

52. A polypeptide encoded by the polynucleotide of any of claims 45-48 or the vector of claim 51.

53. A method of generating an engineered cell comprising introducing into the cell the polynucleotide of claims 45-48, the vector of claim 51, or the polypeptide of claim 52.

54. An engineered cell comprising the polynucleotide of any of claims 45-48, the vector of claim 51, or the polypeptide of claim 52.

55. The engineered cell of claim 54, wherein the engineered cell is a T cell.

56. The engineered cell of claim 54, wherein the engineered cell is a natural killer (NK) cell.

57. A population of engineered cells comprising the engineered cell of any of claims 54-56.

58. A pharmaceutical composition comprising the population of engineered cells of claim 57 and an excipient.

59. A method for treating a subject having cancer, the method comprising administering to the subject a therapeutically effective amount of the pharmaceutical composition of claim 58.

60. The method of claim 59, wherein the subject has a B-cell malignancy.

61. The method of claim 60, wherein the subject has diffuse large B-cell lymphoma, high-grade B-cell lymphoma, follicular lymphoma, marginal zone lymphoma, mantle cell lymphoma, Burkitt lymphoma, lymphoplasmacytic lymphoma, hairy cell leukemia, or chronic lymphocytic leukemia.

62. The method of any of claims 59-61, wherein the subject has CD19⁺CD79b⁺ cancer.

63. The method of any of claims 59-61, wherein the subject has CD19⁻CD79b⁺ cancer.

64. The method of any of claims 59-61, wherein the subject has CD19+CD79b cancer.

65. A polynucleotide encoding a polypeptide having SEQ ID NO:15.

66. The polynucleotide of claim 65, wherein the polynucleotide comprises a sequence having at least 90% identity with SEQ ID NO:30.

67. The polynucleotide of claim 66, wherein the polynucleotide comprises SEQ ID NO: 30.

68. A vector comprising the polynucleotide of claim 65-67.

69. An engineered cell comprising the polynucleotide of any of claims 65-67 or the vector of claim 68.

70. The engineered cell of claim 69, wherein the engineered cell is a T cell.

71. The engineered cell of claim 69, wherein the engineered cell is a natural killer (NK) cell.

72. The engineered cell of claim 69, wherein the engineered cell is a macrophage.

73. A population of engineered cells comprising the engineered cell of any of claims 69-71.

74. A pharmaceutical composition comprising the population of engineered cells of claim 73 and an excipient.

75. A method for treating a subject having cancer, the method comprising administering to the subject a therapeutically effective amount of the pharmaceutical composition of claim 74.

76. The method of claim 75, wherein the subject has a B-cell malignancy.

77. The method of claim 76, wherein the subject has diffuse large B-cell lymphoma, high-grade B-cell lymphoma, follicular lymphoma, marginal zone lymphoma, mantle cell lymphoma, Burkitt lymphoma, lymphoplasmacytic lymphoma, hairy cell leukemia, or chronic lymphocytic leukemia.

78. The method of any of claims 75-77, wherein the subject has CD19⁺CD79b⁺ cancer.

79. The method of any of claims 75-77, wherein the subject has CD19 CD79b⁺ cancer.

80. The method of any of claims 75-77, wherein the subject has CD19⁺CD79b⁻ cancer.
